(19) United States
(12) Patent Application Publication
(10) Pub. No.: US 2024/0183865 A1
(43) Pub. Date: Jun. 6, 2024

(54) METHOD FOR MEASURING AGGREGATING PROTEINS, METHOD FOR VISUALIZING AGGREGATION, AND KIT USED THEREFOR

(71) Applicants: KANEKA CORPORATION, Osaka-shi, Osaka (JP); MURORAN INSTITUTE OF TECHNOLOGY, Muroran-shi, Hokkaido (JP)

(72) Inventors: Naoki NISHISHITA, Kobe-shi (JP); Akira KOBAYASHI, Kobe-shi (JP); Kiyotaka TOKURAKU, Muroran-shi (JP); Koji UWAI, Muroran-shi (JP); Masahiro KURAGANO, Muroran-shi (JP)

(73) Assignees: KANEKA CORPORATION, Osaka-shi, Osaka (JP); MURORAN INSTITUTE OF TECHNOLOGY, Muroran-shi, Hokkaido (JP)

(21) Appl. No.: 18/285,090

(22) PCT Filed: Mar. 31, 2022
(86) PCT No.: PCT/JP2022/016478

§ 371 Date: Sep. 29, 2023

(30) Foreign Application Priority Data
Mar. 31, 2021 (JP) 2021-061500
Mar. 31, 2021 (JP) 2021-062006

(51) Int. Cl. G01N 33/68; C07K 1/34; G01N 33/58

(52) U.S. Cl. CPC G01N 33/6896; C07K 1/34; G01N 33/58; G01N 2333/4712; G01N 2333/4713; G01N 2333/76

(57) ABSTRACT

The objective of the present invention is to provide a method for visualizing an aggregate of aggregating proteins in an environment similar to in vivo conditions. More specifically, the present invention relates to a method for collecting an aggregating protein-containing solution by allowing an aggregating protein-containing solution to pass through a filtration filter through which a solution for accelerating and visualizing protein aggregation has passed; a method for visualizing an aggregate of aggregating proteins comprising visualizing an aggregate of aggregating proteins in the aggregating protein-containing solution collected by the collecting method; and a method of forming an aggregate of labeled aggregating proteins of interest in a medium or culture supernatant from which an aggregation inhibitor of 40 kDa or higher has been removed, so as to visualize aggregation.

### TECHNICAL FIELD

The present invention relates to, for example, a method for collecting a solution containing aggregating proteins, a method for measuring the aggregated amount of aggregating proteins, a method for visualizing an aggregate of aggregating proteins, and a kit used for such methods.

### BACKGROUND ART

Alzheimer's disease (it is often abbreviated to “AD” herein) is a type of irreversible progressive central neurological disease associated with symptoms, such as cognitive impairment (dementia), behavioral disorders, or personality change. While the number of patients with dementia is deduced to be 50,000,000 or greater in 2019 all over the world, approximately 70% of such patients are considered to be patients with AD, and an incidence thereof is increasing. Because of increased medical expenses or problems of nursing care resulting from an increasing number of AD patients, economical or emotional burdens are imposed on the nations or persons involved in patients, and, accordingly, such problem is a serious issue of concern of the society in recent years.

AD is developed as follows. Hydrophobic peptides, amyloid β proteins (often referred to as “Aβ” herein), are aggregated and accumulated in the patient's brain, microtubule-binding proteins, tau proteins, are hyperphosphorylated to become fibrotic, neurons are destroyed, and the brain is then shrunk (Non-Patent Documents 1 to 3).

On the basis of the mechanism of AD development, a technique of evaluating effects of a test substance added in vitro for inhibiting Aβ aggregation and screening for a candidate compound for treatment of AD has been developed. For example, a method of microliter-scale high throughput screening (which is abbreviated to “MSHTS” herein) of an amyloid β protein aggregation inhibitor using a quantum dot nanoprobe is a cell-free assay-based screening technique that can search for a candidate compound having effects of inhibiting amyloid β protein aggregation in a PBS solvent (Non-Patent Document 4).

As an example of MSHTS, for example, Patent Document 1 discloses a method, an apparatus, and a program for evaluating amyloid formation. Specifically, Patent Document 1 discloses a method for determining inhibitory activity of a test substance on amyloid formation comprising: a step of aggregation comprising allowing an amyloid forming protein, such as an amyloid β protein, to react with a fluorescent probe (e.g., a probe comprising a quantum dot as a fluorescent dye or a quantum dot) capable of binding to an amyloid formed as a result of polymerization of the amyloid forming protein in an adequate buffer such as water or PBS in the presence or absence of the test substance; a step of imaging comprising obtaining an image of a fluorescence of the aggregation product obtained in the step of aggregation; a step of calculating a standard deviation based on the luminance value of pixels included in an area of interest in the fluorescent image obtained in the step of imaging; and a step of activity evaluation to determine that a test substance has inhibitory activity on amyloid formation when the standard deviation of the luminance in the presence of the test substance is smaller than the standard deviation of the luminance in the absence of the test substance as a result of comparison between the standard deviation of the luminance in the presence of the test substance and the standard deviation of the luminance in the absence of the test substance calculated in the step of calculating a standard deviation.

Patent Document 2 discloses a universal quantum dot nanoprobe used for evaluating amyloid aggregating properties of a protein or peptide and a method for evaluating an amyloid formation inhibitor using such quantum dot nanoprobe. Specifically, Patent Document 2 discloses a quantum dot nanoprobe comprising quantum dots bound to the N- or C-terminus of the amyloid forming peptide via cysteine.

However, inhibitory effects of a candidate compound selected by the cell-free assay system are not observed in the cell-based assay system (the test system using cells) in many occasions. This is considered to occur because the cell-free assay system does not necessarily reflect the in vivo environment. If MSHTS can be performed in a medium more similar to the in vivo environment, a candidate compound or a candidate material that can exert inhibitory activity in vivo can be screened accurately and efficiently. When quantum dots are added to a medium or the like, however, new problem occurs, i.e., Aβ would not aggregate. Therefore, it was impossible to use MSHTS as a screening system to search for a candidate compound in an environment more similar to the in vivo environment.

### PRIOR ART DOCUMENTS

### Patent Documents

Patent Document 1: WO 2020/138265
Patent Document 2: JP 2017-007990 A

### Non-Patent Documents

Non-Patent Document 1: Hardy J. and Selkoe D. J., 2002, Science, 297 (5580): 353-356
Non-Patent Document 2: Jack C. R. Jr., et al., 2010, Lancet Neurol., 9 (1): 119-128
Non-Patent Document 3: Akira Tamaoka, 2017, Proceedings of the Annual Meeting of the Japanese Research Group on Senile Dementia, Vol. 22, No. 3, pp. 19-23
Non-Patent Document 4: Ishigaki et al., 2013, PLOS ONE, 8 (8): e72992

### SUMMARY OF THE INVENTION

### Objects to be Attained by the Invention

Under the above circumstances, it is an object of the present invention to provide a method for visualizing an aggregate of aggregating proteins, such as AP, in an environment similar to in vivo conditions.

### Means for Attaining the Objects

Through finding a tool that constitutes a technique of visualizing an aggregate of aggregating proteins (e.g., amyloid β proteins) in an environment more similar to in vivo conditions than PBS (e.g., a medium in which cells can be cultured) and development thereof, a compound that can exert its effects in vivo can be found more precisely.

Unlike an environment such as in PBS, an aggregate of aggregating proteins (e.g., amyloid β proteins) is formed in the presence of a variety of contaminants under conditions similar to in vivo environment (e.g., a medium or culture supernatant). The present inventors deduced the presence of some contaminants that would inhibit aggregation of aggregating proteins including amyloid β proteins in a medium or culture supernatant and searched for such contaminants. As a result, they found that aggregation of amyloid β proteins would be inhibited and aggregation would not be visualized when a medium or culture supernatant contained a contaminant of 40 kDa or higher (e.g., an actin protein (which is often denoted as “actin” herein), an albumin protein (which is often denoted as “albumin” herein), and globulin). Therefore, a contaminant of 40 kDa or higher that would inhibit aggregation was removed from a medium using an ultrafiltration filter or the like, the amyloid β proteins were allowed to aggregate again in the medium, and, in such a case, it was found possible to visualize an aggregate. In an aspect, the present invention is based on such new finding.

When a contaminant is removed from a medium, aggregating proteins contained in the medium disappear in the step of removing a contaminant, and the total amount of target proteins to be collected is reduced, disadvantageously.

The present inventors have conducted concentrated studies in order to solve the problems as described above. As a result, they found that use of a solution for accelerating and visualizing protein aggregation would enable reduction of protein adsorption in the step of removing a contaminant, efficient collection of aggregating proteins, and visualization of an aggregate of aggregating proteins in a collected solution (a filtrate). This has led to the completion of the present invention.

Specifically, the present invention includes the following.

(1) A method for collecting an aggregating protein-containing solution comprising: a first step of allowing a solution for accelerating and visualizing protein aggregation to pass through a filtration filter; and a second step of allowing an aggregating protein-containing solution to pass through the filtration filter through which the solution for accelerating and visualizing protein aggregation has passed.
(2) The method according to (1), which further comprises, before the first step, a step of pretreatment for allowing a wash solution to pass through a filtration filter.
(3) The method according to (1) or (2), which further comprises, before the second step, a step of quantifying aggregating proteins in an aggregating protein-containing solution.
(4) The method according to any one of (1) to (3), which further comprises, after the second step, a step of quantifying aggregating proteins in a filtrate.
(5) The method according to any one of (1) to (4), wherein the filtration filter is an ultrafiltration filter with a nominal molecular weight limit (NMWL) of 50 kDa or lower.
(6) The method according to any one of (1) to (5), wherein the solution for accelerating and visualizing protein aggregation comprises a surface-active substance of 50 kDa or lower.
(7) The method according to (6), wherein the surface-active substance is a surfactant.
(8) The method according to (7), wherein the surfactant is a nonionic surfactant.
(9) The method according to (8), wherein the nonionic surfactant is at least one surfactant selected from among an ester-type nonionic surfactant, an ether-type nonionic surfactant, an ester- and ether-type nonionic surfactant, and an alkylglycoside-type nonionic surfactant.
(10) The method according to (9), wherein the nonionic surfactant is an ester- and ether-type nonionic surfactant.
(11) The method according to (10), wherein the ester- and ether-type nonionic surfactant is Tween 20.
(12) The method according to (11), wherein the solution for accelerating and visualizing protein aggregation is an aqueous solution containing Tween 20.
(13) The method according to (12), wherein the concentration of Tween 20 is 0.1% to 10% in the aqueous solution.
(14) The method according to any one of (1) to (13), wherein the aggregating protein-containing solution comprises a buffer, medium, or culture supernatant.
(15) The method according to any one of (1) to (14), wherein the aggregating protein-containing solution comprises a cell metabolite, cell secretory factor, inorganic substance, or organic acid.
(16) The method according to any one of (1) to (15), wherein the aggregating protein-containing solution comprises a disease-associated protein.
(17) The method according to (16), wherein the disease is Alzheimer's disease and the protein associated therewith is the amyloid β protein or tau protein.
(18) The method according to any one of (1) to (15), wherein the aggregating protein-containing solution comprises at least one substance selected from among tau protein, α-synuclein, amyloid β protein, prion protein, TDP-43, polyglutamic acid, Atg-8, and Atg-15.
(19) The method according to any one of (1) to (18), wherein a rate of aggregating protein collection determined by the equation 1) below is over 60%.

Rate of aggregating protein collection (%)=quantitative value of aggregating proteins after second step/quantitative value of aggregating proteins before second step×100 Equation 1)

(20) The method according to any one of (1) to (19), wherein the aggregating protein is the amyloid β protein.
(21) The method according to (20), wherein the amyloid β protein is the amyloid β42 protein.
(22) A method for measuring an aggregated amount of aggregating proteins comprising measuring an aggregated amount of the aggregating proteins in the aggregating protein-containing solution collected by the method according to any one of (1) to (21).
(23) The method according to (22), wherein the aggregating protein is at least one protein selected from among tau protein, α-synuclein, amyloid β protein, prion protein, TDP-43, polyglutamic acid, Atg-8, and Atg-15.
(24) The method according to (22), wherein a quantum-dot-modified amyloid β protein is added to the solution containing the amyloid β protein collected by the method of (20), and an aggregate of the amyloid β protein and the quantum-dot-modified amyloid β protein is measured with the use of the quantum dot as an indicator.
(25) The method according to (24), wherein the amyloid β protein is the amyloid β42 protein.
(26) A method for visualizing an aggregate of aggregating proteins comprising visualizing an aggregate of aggregating proteins in the aggregating protein-containing solution collected by the method according to any one of (1) to (21).
(27) The method according to (26), wherein visualization of an aggregate of aggregating proteins comprises: a step of aggregation comprising incubating the entirely or partially labeled aggregating proteins and/or aggregating fragments thereof in the collected aggregating protein-containing solution and allowing the aggregating proteins and/or aggregating fragments thereof to aggregate; and a step of detection comprising detecting an aggregate of the aggregating proteins and/or aggregating fragments thereof, wherein, from the collected aggregating protein-containing solution, an aggregation inhibitor of 40 kDa or higher that inhibits aggregation of the aggregating proteins and/or aggregating fragments thereof is removed.
(28) The method according to (27), wherein the aggregation inhibitor of 40 kDa or higher has a molecular weight of 250 kDa or lower.
(29) The method according to (27) or (28), wherein the aggregation inhibitor of 40 kDa or higher is at least one polypeptide selected from the group consisting of actin, albumin, and globulin or a fragment thereof.
(30) The method according to any one of (27) to (29), wherein the label is an optical label.
(31) The method according to (30), wherein the optical label is a quantum dot.
(32) The method according to any one of (26) to (31), wherein the aggregating protein is at least one protein selected from among tau protein, α-synuclein, amyloid β protein, prion protein, TDP-43, polyglutamic acid, Atg-8, and Atg-15.
(33) The method according to any one of (26) to (32), wherein a quantum-dot-modified amyloid 3 protein is added to the solution containing the amyloid 1 protein collected by the method of (20), and an aggregate of the amyloid β protein and the quantum-dot-modified amyloid β protein is visualized based on the quantum dot.
(34) The method according to (33), wherein the amyloid β protein is the amyloid β42 protein.
(35) A kit for collecting aggregating proteins from an aggregating protein-containing solution by the method according to any one of (1) to (21), which comprises a filtration filter and a solution for accelerating and visualizing protein aggregation.
(36) A kit for measuring an aggregated amount of the aggregating proteins in an aggregating protein-containing solution by the method according to any one of (22) to (25), which comprises a filtration filter and a solution for accelerating and visualizing protein aggregation.
(37) A kit for visualizing an aggregate of the aggregating proteins in an aggregating protein-containing solution by the method according to any one of (26) to (34), which comprises a filtration filter and a solution for accelerating and visualizing protein aggregation.
(38) The kit according to (37), which further comprises a means for labeling aggregating proteins or labeled aggregating proteins.
(39) The kit according to any one of (35) to (38), which further comprises a wash solution.
(40) The kit according to any one of (35) to (39), which further comprises a means for aggregating protein quantification.
(41) The kit according to any one of (35) to (40), wherein the filtration filter is an ultrafiltration filter with a nominal molecular weight limit (NMWL) of 50 kDa or lower.
(42) The kit according to any one of (35) to (41), wherein the solution for accelerating and visualizing protein aggregation comprises a surface-active substance of 50 kDa or lower.
(43) The kit according to (42), wherein the surface-active substance is a surfactant.
(44) The kit according to (43), wherein the surfactant is a nonionic surfactant.
(45) The kit according to (44), wherein the nonionic surfactant is at least one surfactant selected from among an ester-type nonionic surfactant, an ether-type nonionic surfactant, an ester- and ether-type nonionic surfactant, and an alkylglycoside-type nonionic surfactant.
(46) The kit according to (45), wherein the nonionic surfactant is an ester- and ether-type nonionic surfactant.
(47) The kit according to (46), wherein the ester- and ether-type nonionic surfactant is Tween 20.
(48) The kit according to (47), wherein the solution for accelerating and visualizing protein aggregation is an aqueous solution containing Tween 20.
(49) The kit according to (48), wherein the concentration of Tween 20 is 0.1% to 10% in the aqueous solution.
(50) The kit according to any one of (35) to (49), wherein the aggregating protein-containing solution comprises a buffer, medium, or culture supernatant.
(51) The kit according to any one of (35) to (50), wherein the aggregating protein-containing solution comprises a cell metabolite, cell secretory factor, inorganic substance, or organic acid.
(52) The kit according to any one of (35) to (51), wherein the aggregating protein-containing solution comprises at least one substance selected from among tau protein, α-synuclein, amyloid β protein, prion protein, TDP-43, polyglutamic acid, Atg-8, and Atg-15.
(53) The kit according to any one of (35) to (52), wherein the aggregating protein is the amyloid D protein.
(54) The kit according to (53), wherein the amyloid β protein is the amyloid β42 protein.
(55) The kit according to any one of (36) to (54), which further comprises a quantum-dot-modified amyloid β protein.

In addition, the present invention includes the following.

(1′) A method for visualizing aggregation of aggregating proteins comprising: a step of aggregation comprising incubating the entirely or partially labeled aggregating proteins and/or aggregating fragments thereof in an aggregating protein-containing solution and allowing the aggregating proteins and/or aggregating fragments thereof to aggregate; and a step of detection comprising detecting an aggregate of the aggregating proteins and/or aggregating fragments thereof, wherein, from the aggregating protein-containing solution, an aggregation inhibitor of 40 kDa or higher that inhibits aggregation of the aggregating proteins and/or aggregating fragments thereof is removed.
(2′) The method for visualizing aggregation according to (I′), wherein the aggregation inhibitor of 40 kDa or higher has a molecular weight of 250 kDa or lower.
(3′) The method for visualizing aggregation according to (1′) or (2′), wherein the aggregation inhibitor of 40 kDa or higher is at least one polypeptide selected from the group consisting of actin, albumin, and globulin or a fragment thereof.
(4′) The method for visualizing aggregation according to (1′) or (2′), wherein the step of aggregation comprises a step of removal for removing the aggregation inhibitor from an aggregating protein-containing solution.
(5′) The method for visualizing aggregation according to (4′), wherein the step of removal is performed via filtration, adsorption, centrifugation, or a combination of any thereof.
(6′) The method for visualizing aggregation according to (4′) or (5′), wherein the step of removal is filtration performed with the use of an ultrafiltration filter with a nominal molecular weight limit (NMWL) of 50 kDa or lower.
(7′) The method for visualizing aggregation according to any one of (1′) to (6′), wherein a solvent of the aggregating protein-containing solution is a medium or culture supernatant.
(8′) The method for visualizing aggregation according to any one of (1′) to (7′), wherein the label is an optical label.
(9′) The method for visualizing aggregation according to (8′), wherein the optical label is a quantum dot.
(10′) The method for visualizing aggregation according to any one of (1′) to (9′), wherein the aggregating protein is a disease-associated protein.
(11′) The method for visualizing aggregation according to (10′), wherein the disease is selected from the group consisting of Alzheimer's disease, Parkinson's disease, amyotrophic lateral sclerosis, Huntington's disease, prion disease, diabetes, and arterial sclerosis.
(12′) The method for visualizing aggregation according to (10′), wherein the disease is Alzheimer's disease and the protein associated therewith is the amyloid β protein or tau protein.
(13′) An agent for visualizing aggregation consisting of a medium or culture supernatant from which an aggregation inhibitor of 40 kDa or higher or an aggregation inhibitor consisting of at least one polypeptide selected from the group consisting of actin, albumin, and globulin or a fragment thereof is removed, wherein the agent is capable of visualizing aggregation of aggregating proteins.
(14′) The agent for visualizing aggregation according to (13′), wherein the aggregation inhibitor of 40 kDa or higher has a molecular weight of 250 kDa or lower.
(15′) The agent for visualizing aggregation according to (13′) or (14′), wherein the aggregation inhibitor of 40 kDa or higher is at least one polypeptide selected from the group consisting of actin, albumin, and globulin or a fragment thereof.
(16′) A kit for visualizing aggregation of aggregating proteins comprising a means for removing an aggregation inhibitor of 40 kDa or higher and/or a means for labeling aggregating proteins or labeled aggregating proteins.
(17′) The kit for visualizing aggregation according to (16′), wherein the aggregation inhibitor of 40 kDa or higher has a molecular weight of 250 kDa or lower.
(18′) The kit for visualizing aggregation according to (16′) or (17′), wherein the aggregation inhibitor of 40 kDa or higher is at least one polypeptide selected from the group consisting of actin, albumin, and globulin or a fragment thereof.
(19′) The kit for visualizing aggregation according to any one of (16′) to (18′), wherein the label is an optical label.
(20′) The kit for visualizing aggregation according to (19′), wherein the optical label is a quantum dot.
(21′) The kit for visualizing aggregation according to any one of (16′) to (20′), wherein the aggregating protein is a disease-associated protein.
(22′) The kit for visualizing aggregation according to (21′), wherein the disease is selected from the group consisting of Alzheimer's disease, Parkinson's disease, amyotrophic lateral sclerosis, Huntington's disease, prion disease, diabetes, and arterial sclerosis.
(23′) The kit for visualizing aggregation according to (21′), wherein the disease is Alzheimer's disease and the protein associated therewith is the amyloid D protein or tau protein.
(24′) A method for production characterized in that an aggregate is formed of aggregating proteins and/or aggregating fragments thereof comprising: a step of aggregation comprising incubating the entirely or partially labeled aggregating proteins in an aggregating protein-containing solution from which an aggregation inhibitor of 40 kDa or higher is removed and allowing the aggregating proteins and/or aggregating fragments thereof to aggregate: and a step of detecting the aggregated aggregating proteins and/or aggregating fragments thereof to verify formation of an aggregate.
(25′) The method of production according to (24′), wherein the aggregation inhibitor of 40 kDa or higher has a molecular weight of 250 kDa or lower.
(26′) The method of production according to (24′) or (25′), wherein the aggregation inhibitor of 40 kDa or higher is at least one polypeptide selected from the group consisting of actin, albumin. and globulin or a fragment thereof.

This description includes part or all of the content as disclosed in the descriptions and/or drawings of Japanese Patent Application Nos. 2021-061500 and 2021-062006, which are priority documents of the present application.

### Effects of the Invention

According to the present invention, aggregating proteins can be efficiently collected from an aggregating protein-containing solution while removing contaminants from the solution, and an aggregate of aggregating proteins can be visualized in the collected solution. According to the present invention, in addition, aggregating proteins can be aggregated in a medium or culture supernatant and aggregation thereof can be visualized.

### BRIEF DESCRIPTION OF THE DRAWINGS

FIG. 1 shows the residual ratio of the artificial amyloid β42 protein (Aβ₄₂) after ultrafiltration determined in Examples. The residual ratio was calculated based on the residual ratio of artificial Aβ₄₂ in a “simulated solution” designated as 100%.

FIG. 2 shows the residual ratio of artificial Aβ₄₂ after ultrafiltration determined in Examples. The residual ratio was calculated based on the residual ratio of artificial Aβ₄₂ in a “simulated solution” designated as 100%. FIG. 2 demonstrates that use of a Blocking 2 composition enabled collection of Aβ₄₂ in the solution with the highest efficiently. Blocking 1 was prepared by allowing a 5% BSA-containing PBS solution to stand at room temperature (15° C. to 25° C.) for 2 hours, Blocking 2 was prepared by allowing a 5% BSA-containing PBS solution supplemented with Tween 20 to the final concentration of 0.10% to stand at room temperature (15° C. to 25° C.) for 2 hours, Blocking 3 was prepared by allowing a 5% BSA-containing PBS solution to stand at 4° C.±2° C. for 18 hours, Blocking 4 was prepared by allowing a 5% BSA-containing PBS solution supplemented with Tween 20 to the final concentration of 0.1% to stand at 4° C.±2° C. for 18 hours, Blocking 5 was prepared by allowing a 0.4% BSA-containing PBS solution to stand at 4° C.±2° C. for 18 hours, and Blocking 6 was prepared by allowing a 0.4% BSA-containing PBS solution supplemented with Tween 20 to the final concentration of 0.1% to stand at 4° C.±2° C. for 18 hours.

FIG. 3 shows the residual ratio of the secreted amyloid β protein (Aβ) determined in Examples. The residual ratio was calculated based on the residual ratio of secreted AP in a “culture supernatant” designated as 100%. FIG. 3 demonstrates that use of Blocking 2 enabled reduction in removal of Aβ from the culture supernatant.

FIG. 4 shows the results of evaluation as to whether or not use of Blocking 2 enables visualization of aggregated Aβ by MSHTS using the filtrate after ultrafiltration and quantitative analysis of an AP aggregation inhibiting material performed in Examples. “Control” indicates dimethyl sulfoxide (DMSO) and “RA” indicates rosmarinic acid. FIG. 4 demonstrate that use of Blocking 2 did not enable visualization of aggregated Aβ in the filtrate after ultrafiltration via MSHTS. Since the composition of Blocking 2 may adversely affect, a solution that can be subjected to MSHTS and can reduce removal of Aβ from the culture supernatant was selected again.

FIG. 5 shows the results attained with the use of a solution for accelerating and visualizing protein aggregation (Buffer 12) that enables visualization of aggregated Aβ by MSHTS while reducing removal of Aβ from the culture supernatant and enables searching of an Aβ aggregation inhibiting material in Examples. Buffer 8 is diluted BSA containing Tween 20, Buffer 9 is diluted BSA, Buffer 10 is diluted B-27 containing Tween 20, Buffer 11 is diluted B-27, and Buffer 12 is a 0.1% Tween 20 solution (a solution prepared by adjusting Tween 20 to the final concentration of 0.1% in PBS).

FIG. 6 shows formation of Aβ aggregates depending on types of solvents: a: a fluorescent image at the initiation of aggregation (0 hour) with the use of PBS as a solvent: b: a fluorescent image obtained 24 hours after the initiation of incubation of the solution of a; c: a fluorescent image obtained 24 hours after the initiation of incubation of a solution of PBS supplemented with BSA; d: a fluorescent image obtained 24 hours after the initiation of incubation using a medium comprising a neurobasal medium supplemented with B-27 at 0.5% v/v (NBM medium) as a solvent: and e: a fluorescent image obtained 24 hours after the initiation of incubation using DFBM comprising DMEM/F12 medium supplemented with B-27 as a solvent. Fluorescent images at the initiation of aggregation (0 hour) using PBS/BSA, NBM, and DFBM are not different from c, d, and e, respectively and thus are not shown.

FIG. 7 shows formation of Aβ aggregates when the media after ultrafiltration with filters of various sizes were used as solvents: a: a fluorescent image at the initiation of aggregation (0 hour) when the NBM medium after ultrafiltration with a 3 kDa filter was used as a solvent: b: a fluorescent image obtained 24 hours after the initiation of incubation of the solution of a: c: a fluorescent image obtained 24 hours after the initiation of incubation when the NBM medium after ultrafiltration with a 10 kDa filter was used as a solvent: d: a fluorescent image obtained 24 hours after the initiation of incubation when the NBM medium after ultrafiltration with a 50 kDa filter was used as a solvent; and e: a fluorescent image obtained 24 hours after the initiation of incubation when the NBM medium after ultrafiltration with a 100 kDa filter was used as a solvent.

FIG. 8 shows SDS-PAGE to evaluate Aβ aggregation inhibitors. In each lane in FIG. 8, an electrophoresis sample indicated by a corresponding number in the figure was subjected to electrophoresis. In the figure, a band at around 42 kDa indicated by an arrow represents actin, a band at around 50 kDa indicated by an arrow represents albumin, and a band at around 160 kDa indicated by an arrow represents globulin.

FIG. 9 shows the areas, the perimeters, and the Feret's diameters of randomly selected 10 aggregates determined to be composed of single particles via image analysis of Aβ aggregates formed 24 hours later in the filtrates collected after filtration through various filters in FIG. 7: A: areas of aggregates; B: perimeters of aggregates: and C: Feret's diameters of the aggregates. In tables, PBS corresponds to the aggregate shown in FIG. 6 b, 3k corresponds to the aggregate shown in FIG. 7 b, 10k corresponds to the aggregate shown in FIG. 7 c, 50k corresponds to the aggregate shown in FIG. 7 d, and 100k corresponds to the aggregate shown in FIG. 7 e.

FIG. 10 shows the concept of a screening technique using the present invention.

### EMBODIMENTS OF THE INVENTION

Hereafter, the present invention is described in detail.

1. Method for collecting aggregating protein-containing solution

### 1-1. Outline

The first aspect of the present invention relates to a method for collecting an aggregating protein-containing solution. The method of the present aspect comprises a first step of allowing a solution for accelerating and visualizing protein aggregation to pass through a filtration filter: and a second step of allowing an aggregating protein-containing solution to pass through the filtration filter through which the solution for accelerating and visualizing protein aggregation has passed. According to the method of the present aspect, aggregating proteins can be efficiently collected while removing a contaminant (an aggregation inhibitor) from an aggregating protein-containing solution.

### 1-2. Definitions of Terms

The following terms used herein are defined. Unless otherwise specified, the definitions of the terms below are used in common in other aspects of the present invention.

### (1) Filtration Filter

A “filtration filter” is a filter that can fractionate a substance of a given molecular weight by filtration. An example thereof is an ultrafiltration filter. While a type of an ultrafiltration filter is not limited, in the present invention, it is preferable to use an ultrafiltration filter having a nominal molecular weight limit (NMWL) that can remove an aggregation inhibitor of 40 kDa or higher that inhibits aggregation of aggregating proteins. In general, many ultrafiltration filters are known to be capable of filtering a substance with a molecular weight somewhat lower than NMWL. For example, a filtration filter of 50 kDa (UFC505096. Merck) is known to be capable of fractionating approximately 90% of a BSA solution of 67 kDa (1 mg/ml) and approximately a half (approximately 65% with a swing bucket rotor, approximately 55% with a fixed-angle rotor) of an ovalbumin solution (1 mg/ml) of a molecular weight of 45 kDa (Amicon® Ultra-15 Centrifugal Filter Devices User Guide). If NMWL is 50 kDa, specifically, it is substantially possible to remove a protein of 40 kDa or higher. Accordingly, an ultrafiltration filter with NMWL of 50 kDa or lower is preferable as a filtration filter used in the present invention, although a filter is not limited thereto. An example of an ultrafiltration filter with NMWL of 50 kDa or lower is an Amicon Ultra-0.5, Ultracel-50 Membrane, 50 kDa (UFC505096, Millipore).

### (2) Aggregation Inhibitor

An “aggregation inhibitor” is a substance that inhibits aggregation of aggregating proteins. In the present invention, an aggregation inhibitor of 40 kDa or higher falls under the category thereof. When it is simply denoted as an “aggregation inhibitor” herein, accordingly, the term refers to an aggregation inhibitor of 40 kDa or higher, unless otherwise specified. An aggregation inhibitor of 40 kDa or higher is not particularly limited, as long as such substance has a molecular weight of 40 kDa or higher, 41 kDa or higher, or 42 kDa or higher. An aggregation inhibitor of 40 kDa or higher is a substance having a molecular weight of, for example, 250 kDa or lower, 240 kDa or lower, 230 kDa or lower, 220 kDa or lower, or 210 kDa or lower. A specific example of the aggregation inhibitor of 40 kDa or higher is an aggregation inhibitor consisting of at least one polypeptide selected from the group consisting of an actin protein, albumin, and globulin or a fragment thereof.

When an aggregating protein-containing solution contains an aggregation inhibitor in a particular amount or more, aggregating proteins cannot form an aggregate. Accordingly, the presence of an aggregation inhibitor would inhibit visualization of an aggregate of aggregating proteins. The present invention is most characterized in that aggregating proteins are allowed to aggregate with the use of an aggregating protein-containing solution from which an aggregation inhibitor has been removed. When an “aggregation inhibitor has been removed” herein, an aggregation inhibitor is to be removed from an aggregating protein-containing solution (a medium or culture supernatant) by, for example, the method described with regard to the step of removal below or an aggregation inhibitor has already been removed without the step of removal; that is, an aggregation inhibitor has not been included from the beginning. When the step of removal is performed, it is preferable that the aggregation inhibitor be completely (100%) removed from the aggregating protein-containing solution after the step of removal. If the amount of the aggregation inhibitor is not sufficient to inhibit aggregation of aggregating proteins, the aggregation inhibitor may remain therein. When the amount of the aggregation inhibitor is, for example, 0.5% v/v or lower, 0.4% v/v or lower, 0.3% v/v or lower, 0.2% v/v or lower, or 0.1% v/v or lower in the aggregating protein-containing solution, persistence of the aggregation inhibitor is acceptable.

### (3) Solution for Accelerating and Visualizing Protein Aggregation

A solution for accelerating and visualizing protein aggregation is allowed to pass through a filtration filter to reduce protein adsorption, accelerate aggregation of aggregating proteins in the collected solution, and visualize the aggregation in the step of removal of an aggregation inhibitor using a filtration filter.

An example of a solution for accelerating and visualizing protein aggregation is a solution comprising a surface-active substance of 50 kDa or lower. An example of a surface-active substance is a surfactant. A surfactant is preferably a nonionic surfactant. Examples of nonionic surfactants include an ester-type nonionic surfactant, an ether-type nonionic surfactant, an ester- and ether-type nonionic surfactant, and an alkylglycoside-type nonionic surfactant.

Examples of ester-type nonionic surfactants include allkylsulfate ester salt, polyoxyethylene alkylsulfate ester salt, glycerin fatty acid ester, sorbitan fatty acid ester, sugar fatty acid ester, and fatty acid methyl ester ethoxylate.

Examples of ether-type nonionic surfactants include polyoxyethylene alkyl ether, fatty acid methyl ester ethoxylate, polyoxyethylene alkyl phenyl ether, and polyoxyethylene polyoxypropylene glycol.

Examples of ester- and ether-type nonionic surfactants include Tween 20 (also referred to as Polysorbate 20 or polyethylene glycol sorbitan monolaurate), Tween 40, Tween 60, Tween 80, and Tween 85.

Examples of alkyl glycoside-type nonionic surfactants include n-octyl-β-D-glucoside, n-octyl-β-D-maltoside, n-decyl-β-D-glucoside (decyl glucoside), n-decyl-β-D-maltoside, n-dodecyl-β-D-glucoside (lauryl glucoside), n-heptyl-β-D-thioglucoside, n-octyl-β-D-thioglucoside, and n-nonyl-β-D-thiomaltoside.

A nonionic surfactant is preferably an ester- and ether-type nonionic surfactant, and an ester- and ether-type nonionic surfactant is particularly preferably Tween 20. An example of a solution for accelerating and visualizing protein aggregation is an aqueous solution containing Tween 20. The concentration of Tween 20 in the aqueous solution is, for example, 0.1% to 10%, preferably 0.01% to 1%, and particularly preferably 0.05% to 0.5%.

### (4) Aggregating Proteins

Aggregating proteins assemble to form an aggregate. Types of aggregating proteins are not limited. Examples thereof include polyglutamic acids, disease-associated proteins, and autophagy-associated proteins.

Examples of disease-associated aggregating proteins include causal proteins of Alzheimer's disease, such as the amyloid D protein and the tau protein (including the phosphorylated tau protein), a causal protein of Parkinson's disease, such as the α-synuclein protein, a causal protein of transmissible spongiform encephalopathies (including Creutzfeldt-Jakob disease, bovine spongiform encephalopathies, and prion disease), such as the prion protein, a causal protein of Huntington's disease, such as the huntingtin protein, a causal protein of type II diabetes, such as the amylin protein, a causal protein of arterial sclerosis (including cerebral infarction, pulmonary infarction, and myocardial infarction), such as the apolipoprotein A1 (APOA1 protein), a causal protein of articular rheumatism, such as the serum amyloid A protein, a causal protein of systemic AL amyloidosis, such as the immunoglobulin light chain, a causal protein of dialysis amyloidosis, such as the β2 microglobulin, and a causal protein of amyotrophic lateral sclerosis, such as the TDP-43 protein. In particular, the amyloid β proteins, such as the amyloid β42 protein, the amyloid β43 protein, and the amyloid β38 protein, are preferable.

Examples of autophagy-associated proteins include the ubiquitin-like proteins, such as Atg-8 and Atg-12.

Examples of aggregating proteins include tau protein (such as phosphorylated tau protein), α-synuclein, amyloid β protein, prion protein, TDP-43, polyglutamic acid, Atg-8, and Atg-15, with the amyloid β proteins, such as the amyloid β42 protein, the amyloid β43 protein, and the amyloid β38 protein, being preferable.

Aggregating proteins may be naturally-occurring proteins existing in nature, modified proteins derived from naturally-occurring proteins by artificial variation or modification, or artificial proteins comprising artificially designed amino acid sequences.

The aggregating proteins and aggregating fragments described below are entirely or partially labeled. When “entirely” labeled, all the aggregating proteins or aggregating fragments are labeled. When “partially” labeled, one or more to less than all of the aggregating proteins or aggregating fragments are labeled in the group of aggregating proteins or aggregating fragments.

The term “aggregating fragment” used herein refers to a peptide having a region or domain that can contribute to aggregation of the aggregating proteins and having aggregating activity similar to that of aggregating proteins.

### (5) Aggregate

The term “aggregate” used herein refers to an assembly of two or more aggregating proteins and/or aggregating fragments thereof. A so-called “protein complex” is within the scope of the “aggregate” herein.

An aggregate may be a homoaggregate consisting of a single type of proteins or aggregating fragments thereof or a heteroaggregate consisting of different types of proteins or aggregating fragments thereof.

An aggregate may be a complex having biological functions or a simple assembly.

A diameter (size) of an aggregate is not limited and, in general, it may be within a range of 10 nm to 10 mm. A diameter is preferably 100 nm to 5 mm, and more preferably 500 nm to 1 mm.

### (6) Aggregating Protein-Containing Solution

An aggregating protein-containing solution may be any solution, as long as it comprises aggregating proteins. Examples thereof include physiological saline, a buffer, a medium, and a culture supernatant comprising aggregating proteins. An aggregating protein-containing solution is preferably a medium or culture supernatant similar to in vivo environment. For example, cells that secrete aggregating proteins are cultured, a culture supernatant is collected from the culture product, and the collected culture supernatant can be used as an aggregating protein-containing solution.

Aggregating proteins contained in an aggregating protein-containing solution may be contained in a solvent in advance and/or added thereto.

An aggregating protein-containing solution may comprise, in addition to aggregating proteins, cell metabolites, cell secretory factors, inorganic substance (inorganic metal salts), and organic acids. Examples of cell metabolites include amino acids, such as glutamic acid and aspartic acid, nucleic acids, such as 5′ guanylic acid, antioxidant materials, such as isoascorbic acid, organic acids, such as acetic acid and butyric acid, polyols, such as glycerol, and vitamins, such as vitamin B2 and vitamin A. Examples of cell secretory factors include the neuronal growth regulator 1 (NEGR1), Neurogenin-3, Kallikrein-8/Neuropsin (Cleaved-Val33), Neurofibromin (NF1), Neuroplastin (NPTN), and Neurotrimin (NT). Examples of inorganic substance (inorganic metal salts) include metal salts (e.g., zinc, copper, iron, and selenium), inorganic salts (e.g., Na, K, and Ca), and inorganic ions (e.g., cations: Li⁺, Na⁺, Mg²⁺, K⁺, and Ca²⁺; anions: Cl⁻, Br⁻, NO₂⁻, and SO₄²⁻). Examples of organic acids include succinic acid, pyruvic acid, and lower amines. The concentration of inorganic substance or organic acid is not limited. For example, it may be within the range of 0.001 ng/ml to 1.00 ng/ml or 0.01 ng/ml to 0.10 ng/ml.

### (7) Cell

The target “cell” in the present invention is a cell that produces and/or secrete aggregating proteins.

A cell may be derived from a multicellular organism. An animal-derived cell is preferable, and a mammalian-derived cell is more preferable. Examples of animals include rodents, such as mice, rats, hamsters, and guinea pigs, livestock or pet animals, such as dogs, cats, rabbits, cows, horses, sheep, and goats, and primates, such as humans, rhesus monkeys, gorillas, and chimpanzees. A human-derived cell is particularly preferable.

A cell type is not limited. Examples thereof include a body-tissue-derived cell, a cell derived from a body-tissue-derived cell, a stem cell, a cell differentiated from a stem cell, and a precursor cell thereof.

The term “body tissue” refers to various tissue constituting the body of an organism. Examples thereof include epithelial tissue, connective tissue, muscle tissue, and nerve tissue.

The term “stem cell” refers to a cell having the potential to differentiate into different types of cells and the potential to self-renew. Examples thereof include an adult stem cell and a pluripotent stem cell.

The “adult stem cell” is a stem cell that exists in various tissue in an adult, the terminal differentiation thereof is incomplete, and such stem cell has a certain degree of pluripotency. An adult stem cell is also referred to as a somatic stem cell or tissue stem cell. Examples thereof include a mesenchymal stem cell, a neural stem cell, an intestinal epithelial stem cell, a hematopoietic stem cell, a hair follicle stem cell, and a pigment stem cell.

The term “pluripotent stem cell” refers to a cell having a pluripotency to differentiate into all types of cells constituting an organism and capable of indefinitely growing while maintaining the pluripotency in in vitro culture under adequate conditions. Examples thereof include an embryonic stem (ES) cell, an embryonic germ stem cell, a germ stem cell, and an induced pluripotent stem (iPS) cell.

For example, iPS cells derived from an Alzheimer's disease (AD) patient are induced to differentiate into neural precursor cells and then into nerve cells, and such nerve cells can be used as cells producing and secreting the amyloid β protein, such as the amyloid β42 protein.

### (8) Buffer

The term “buffer” used herein refers to a solution adjusted to maintain pH within a given range in accordance with properties of a target substance to be mixed, such as a cell or protein. Examples thereof include phosphate buffer, HEPES buffer, NaHCO₃/CO₂ buffer, Tris-HCl buffer, and glycine buffer.

### (9) Medium

In the present invention, a “medium” is a liquid or solid substance prepared to culture cells (e.g., cells producing and/or secreting aggregating proteins). In principle, a medium comprises the minimal amount or more ingredients essential for growth and/or maintenance of cells. In the present invention, a medium can serve as a place where aggregation of aggregating proteins takes place in addition to a place for cell culture. A medium may be a basal medium or a special cell culture medium.

A “basal medium” is a medium that constitutes a base of a medium for various animal cells. Culture can be performed in a basal medium, or various culture additives can be added to a basal medium to prepare a medium specific to any of various cells in accordance with purposes (i.e., a special cell culture medium). Examples of basal media that can be used include, but are not particularly limited to, Neurobasal® medium, BME medium, BGJb medium, CMRL1066 medium, Glasgow MEM medium, Improved MEM Zinc Option medium, Iscove's Modified Dulbecco's (IMDM) medium, Medium 199 medium, Eagle MEM medium, αMEM medium, Dulbecco's Modified Eagle's (DMEM) medium, Ham's F10 medium. Ham's F12 medium, RPMI 1640 medium, Fischer's medium, and a medium mixture of any thereof (e.g., DMEM/F12 medium). In addition, media that are used for human iPS cell or human ES cell culture can be preferably used.

A “special cell culture medium” is a medium optimized for culture of special cells with the addition of various supplements to the basal medium or a medium prepared to be capable of inducing cells to differentiate into particular cells. Examples include nerve cell culture media commercialized by various manufacturers. A specific example is “M medium,” which is obtained by adding, as supplements, NGF2.5S and BDNF to a nerve cell culture medium (Sumitomo Bakelite Co., Ltd.) that has been prepared by adding a culture supernatant of primary astroglia cells cultured on a nutritive medium and serum albumin to a basal medium prepared by adding insulin and transferrin to DMEM/F12 (5:5). A culture medium for pluripotent stem cells, such as human iPS cells or human ES cells, is also within the scope of the special cell culture medium.

A medium may or may not contain serum (i.e., a serum-free medium).

### (10) Culture Supernatant

The term “culture supernatant” used herein refers to a culture solution obtained by culturing cells in the medium for a given period of time and removing cells via centrifugation, filtration, or other means. A “given period of time” may be, for example, 6 hours to 4 days, 12 hours to 3 days, 18 hours to 2 days, or 1 day under general culture conditions (e.g., 5% CO₂, 37° C.). In general, a given period of time may be a period of time until cells reach confluency.

In general, a culture supernatant comprises various substances (e.g., nucleic acid, peptide, and low-molecular-weight compound) metabolized, produced and/or secreted by cells.

### (11) Label

The term “labeling” used herein refers to modification of a target substance to identify the target substance. The target substance can be detected or selected easily with certainty by labeling.

Labeling is performed in accordance with a type of a target substance. Since an aggregate of aggregating proteins or aggregating fragments thereof is to be detected herein, the type of the target substance is a peptide. Accordingly, any means of labeling that can directly or indirectly label a peptide is employed. Examples of direct labeling include a method of labeling comprising binding a label substance to aggregating proteins or aggregating fragments thereof and a method of labeling comprising expressing aggregating proteins or aggregating fragments thereof as a fusion protein thereof with a label peptide. An example of indirect labeling is a method of labeling an antibody that specifically recognizes and binds to aggregating proteins or aggregating fragments thereof or an active fragment thereof directly or indirectly via a secondary antibody. A method for binding a label substance is preferable.

A label substance used for peptide labeling is not limited. Examples thereof include an optical label, an enzyme, a radioactive isotope, and a binding low-molecular-weight compound.

The term “optical label” used herein refers to a label with a fluorescent, luminescent, or other substance that emits light, such as visible light, near infrared light, or near-ultraviolet light.

A “fluorescent substance” is a substance that forms an excited state upon absorption of an excited light at a particular wavelength and emits fluorescence when it returns to the ground state. Fluorescent substances encompass a fluorescent dye and a fluorescent protein.

Examples of fluorescent dyes include quantum dot, FITC. Texas, Texas Red®, Alexa Flour 405, Alexa Flour 488, Alexa Flour 647, Alexa Flour 700, Pacific Blue, DyLight 405, DyLight 550, DyLight 650, phycoerythrin-cyanin 5 (PE-Cy5), phycoerythrin-cyanin 7 (PE-Cy7), phycoerythrin (PE), peridinin chlorophyll protein (PerCP), peridinin chlorophyll protein-cyanin 5.5 (PerCP-Cy5.5), cy3, cy5, cy7, FAM, HEX, VIC®, JOE, ROX, TET, Bodipy493, NBD, TAMRA, Quasar® 670, Quasar® 705, Allophycocyanin (APC), congo red, thioflavin T. thioflavin S. Fluorescamine or a derivative thereof, fluorescein or a derivative thereof, azo compound, rhodamine or a derivative thereof, coumarin or a derivative thereof, pyrene or a derivative thereof, and cyanine or a derivative thereof, with a quantum dot being preferable.

A “quantum dot” (which is often referred to as “QD” herein) is a nanoscale semiconductor crystal having quantum mechanics-based optical properties and emitting visible light and fluoresce in a near-infrared region. In general, a quantum dot is 2 nm to 10 nm in diameter and is composed of approximately 10 to 50 atoms. A quantum dot is excellent in properties, such that a large number of fluorescent colors are obtained depending on particle diameters, and fluorescence fading is less likely to occur. Accordingly, application thereof is advancing as a biosensing material and a cell or animal imaging material.

An example of a “fluorescent protein” is GFP.

Examples of “enzymes” include horseradish peroxidase (HRP), alkaline phosphatase (ALP), and glucose oxidase (GOx).

A “radioactive isotope” is an element that releases radiation among isotopes with different mass numbers. An example thereof is ³⁵S.

The term “binding low-molecular-weight compound” used herein refers to a low-molecular-weight compound that binds to a particular binding protein based on affinity. A specific example is biotin that is known as vitamin B7. Biotin has very high affinity to avidin, which is an albumin-derived protein, and a derivative thereof; i.e., streptavidin or neutravidin, and they are strongly bound to each other. With the utilization of such properties, an antibody is labeled with biotin. Thus, another label substance (e.g., a fluorescent dye) labeled with avidin binds to the biotin-labeled antibody, and antibody labeling can be performed.

### (12) Visualization

When “visualization” is intended herein, aggregation of aggregating proteins and/or aggregating fragments thereof is made recognizable visually or under an optical microscope (including a fluorescent microscope and a differential interference microscope). In general, aggregation can be visualized upon formation of an aggregate. Accordingly, visualization of aggregation of aggregating proteins and/or aggregating fragments thereof is synonymous with formation of an aggregate thereof. Examples of the recognition include visual confirmation of a formed aggregate and, when aggregating proteins are labeled, recognition based on the intensity or conditions (e.g., aggregated or spotted) of the label (e.g., a fluorescent or luminescent label).

### 1-3. Method

The method of the present aspect comprises: as essential steps, a first step of allowing a solution for accelerating and visualizing protein aggregation to pass through a filtration filter, and a second step of allowing an aggregating protein-containing solution to pass through the filtration filter through which the solution for accelerating and visualizing protein aggregation has passed: and, as optional steps, before the first step, a step of pretreatment for allowing a wash solution to pass through a filtration filter, before the second step, a step of quantifying aggregating proteins in an aggregating protein-containing solution, and, after the second step, a step of quantifying aggregating proteins in a filtrate.

Hereafter, the steps are described.

### 1-3-1. Step of Pretreatment for Allowing a Wash Solution to Pass Through a Filtration Filter Before the First Step

A wash solution is allowed to pass through a filtration filter, and the filtration filter is washed via centrifugation. This procedure is performed at least once, and preferably two times or more. When sodium hydroxide is used as a wash solution, disadvantageously, a contaminant may turn into a gel at the time of filtration. In addition, aggregating proteins may not be able to form an aggregate in a filtrate obtained by filtration, disadvantageously. Accordingly, examples of wash solutions include pure water, PBS, and physiological saline. Centrifugation is performed, for example, at 15° C. to 25° C. (e.g., room temperature) and 3,000 to 25,000 g (e.g., 14,000 g) for 3 to 15 minutes (e.g., 10 minutes).

### 1-3-2. First Step of Allowing a Solution for Accelerating and Visualizing Protein Aggregation to Pass Through a Filtration Filter

A solution for accelerating and visualizing protein aggregation is allowed to pass through a filtration filter to reduce adsorption of proteins in the aggregating protein-containing solution that is allowed to pass through the filtration filter. A solution for accelerating and visualizing protein aggregation is added dropwise to a filtration filter, and incubation is performed, for example, at 15° C. to 25° C. (e.g., room temperature) for 0.5 to 4 hours (e.g., 2 hours). After incubation, centrifugation is performed, for example, at 15° C. to 25° C. (e.g., room temperature) and 3,000 to 25,000 g (e.g., 14,000 g) for 3 to 15 minutes (e.g., 10 minutes). As with the case of the step of pretreatment described in 1-3-1 above, a wash solution may be allowed to pass through a filtration filter, and the filtration filter may be washed by centrifugation.

### 1-3-3. Step of Quantifying Aggregating Proteins in an Aggregating Protein-Containing Solution Before the Second Step

Before the second step, aggregating proteins in an aggregating protein-containing solution are quantified in advance. An example of a method for quantifying aggregating proteins is an immunoassay technique using an antibody reacting with aggregating proteins. Examples of immunoassay techniques include enzyme immunoassay (ELISA) and immunochromatography.

1-3-4. Second Step of Allowing an Aggregating Protein-Containing Solution to Pass Through the Filtration Filter Through which the Solution for Accelerating and Visualizing Protein Aggregation has Passed

To the filtration filter through which a solution for accelerating and visualizing protein aggregation was allowed to pass after the first step, an aggregating protein-containing solution is added dropwise and filtered therethrough. After dropwise addition, centrifugation is performed, for example, at 15° C. to 25° C. (e.g., room temperature) and 3,000 to 25,000 g (e.g., 14,000 g) for 3 to 60 minutes (e.g., 30 minutes) to collect a filtrate. The collected filtrate comprises aggregating proteins, and an aggregate of aggregating proteins in the filtrate can be visualized with the use of a solution for accelerating and visualizing protein aggregation.

1-3-5. Step of Quantifying Aggregating Proteins in a Filtrate after the Second Step

As with the case of 1-3-3 above, aggregating proteins in the collected filtrate are quantified. Through the quantification, before the second step, the amount of aggregating proteins can be compared with the amount of aggregating proteins in the aggregating protein-containing solution quantified in advance, and a rate of aggregating protein collection can be determined.

Specifically, a rate of aggregating protein collection can be calculated in accordance with the equation 1) below.

Rate of aggregating protein collection (%)=quantitative value of aggregating proteins after second step/quantitative value of aggregating proteins before second step×100 Equation 1)

When the rate of aggregating protein collection determined by the equation 1) is, for example, over 60%, preferably 65% or higher, 70% or higher, and particularly preferably 75% or higher, it is possible to determine that aggregating proteins are collected at a significant level by the method of the present aspect.

### 1-4. Effects

According to the method of the present aspect, aggregating proteins can be collected efficiently. With the use of a solution for accelerating and visualizing protein aggregation, an aggregate of aggregating proteins can be visualized in the collected solution.

### 2. Method for Measuring the Aggregated Amount of Aggregating Proteins or a Method for Visualizing Aggregation of Aggregating Proteins

### 2-1. Outline

The second aspect of the present invention relates to a method for measuring the aggregated amount of aggregating proteins (which is often abbreviated as the “method for measuring aggregated amount” herein) or a method for visualizing aggregation of aggregating proteins (which is often abbreviated as the “method for aggregation visualization” herein). The method for measuring aggregated amount or the method for aggregation visualization of the present aspect involves the use of an aggregating protein-containing solution from which an aggregation inhibitor that inhibits aggregation of aggregating proteins and/or aggregating fragments thereof has been removed to form a visualizable aggregate, and the aggregate is then detected. The method for measuring aggregated amount or the method for aggregation visualization of the present aspect can provide a screening technique for searching for an aggregation inhibitor in an environment more similar to the in vivo environment, such as in a medium or culture supernatant.

For example, the second aspect of the present invention relates to a method for measuring the aggregated amount of aggregating proteins comprising measuring an aggregated amount of the aggregating proteins in the solution (filtrate) containing the aggregating proteins collected by the method of the first aspect or a method for visualizing an aggregate of aggregating proteins comprising visualizing an aggregate of the aggregating proteins in the solution (filtrate) containing the aggregating proteins collected by the method of the first aspect. In the solution (filtrate) containing the aggregating proteins collected by the method of the first aspect, an aggregate of aggregating proteins can be visualized with the use of a solution for accelerating and visualizing protein aggregation. As a result, the aggregated amount of aggregating proteins in a solution (filtrate) containing the collected aggregating proteins can be measured.

### 2-2. Method

The method for measuring aggregated amount or the method for aggregation visualization of the present aspect comprises, as essential steps, a step of aggregation and a step of detection. Hereafter, the steps are described.

### 2-2-1. Step of Aggregation

In “the step of aggregation,” the entirely or partially labeled aggregating proteins and/or aggregating fragments thereof are incubated in an aggregating protein-containing solution to allow the aggregating proteins and/or aggregating fragments thereof to aggregate. This step is characterized in that aggregating proteins and/or aggregating fragments thereof are aggregated in an aggregating protein-containing solution from which an aggregation inhibitor has been removed.

In this step, aggregating proteins and/or aggregating fragments thereof can be added to an aggregating protein-containing solution. Alternatively, an aggregating protein-containing solution may be supplemented with aggregating proteins and/or aggregating fragments thereof in advance.

Incubation conditions are not particularly limited, provided that aggregating proteins and/or aggregating fragments thereof can be aggregated. For example, temperature may be in a range from the melting point of an aggregating protein-containing solution to lower than the denaturation temperature of the aggregating proteins and/or aggregating fragments thereof. Preferably, such temperature is 5° C. to 50° C., 10° C. to 45° C., 15° C. to 42° C., 20° C. to 40° C., 25° C. to 39° C., 30° C. to 38° C., or 35° C. to 37° C. A period of time is not limited, and it may be in a range of 30 minutes to 240 hours (10 days). For example, a period of time may be 30 minutes to 24 hours, 1 hour to 20 hours, 2 hours to 18 hours, 4 hours to 16 hours, 6 hours to 14 hours, 8 hours to 12 hours, 24 hours (1 day) to 240 hours (10 days), 48 hours (2 days) to 216 hours (9 days), 72 hours (3 days) to 192 hours (8 days), 96 hours (4 days) to 168 hours (7 days), or 120 hours (5 days) to 144 hours (6 days).

This step can comprise a step of removal, according to need.

### (1) Step of Removal

In the “step of removal,” an aggregation inhibitor, specifically, an aggregation inhibitor of 40 kDa or higher or at least one polypeptide selected from the group consisting of actin, albumin, and globulin or a fragment thereof is removed from the aggregating protein-containing solution used in this step. The step of removal is an optional step in the step of aggregation.

The step of removal may be the method of the first aspect.

When an aggregating protein-containing solution contains an aggregation inhibitor, the step of aggregation cannot be implemented. In order to implement the step of aggregation, accordingly, it is necessary that the aggregating protein-containing solution to be used in the step of aggregation do not contain an aggregation inhibitor in at least the initiation stage of aggregation. When a solvent of the aggregating protein-containing solution is a medium or culture supernatant, however, the solvent may contain an aggregation inhibitor. In this step, such aggregation inhibitor is removed in advance.

A method for removing an aggregation inhibitor is not limited. Methods known in the art can be employed. For example, filtration, adsorption, centrifugation, or two or more thereof in combination can be employed.

“Filtration” is a method of removal through a filter. In the method of filtration, an aggregating protein-containing solution or a solvent thereof is allowed to pass through a filter, and an aggregation inhibitor is captured by the filter based on its molecular weight to remove the aggregation inhibitor. When the aggregation inhibitor to be removed is an aggregation inhibitor of 40 kDa or higher, as described above, an ultrafiltration filter capable of molecular weight-based fractionation at 40 kDa or lower may be used, so that the present step can be implemented. In the case of an aggregation inhibitor exceeding 40 kDa, the present step can be implemented with the use of an ultrafiltration filter capable of molecular weight-based fractionation at 40 kDa or higher. When the molecular weight of aggregating proteins is significantly different from that of an aggregation inhibitor, this method is preferable. While filtration may be performed by spontaneous dropping, centrifugal filtration comprising filtration in combination with centrifugation enables treatment to be performed within a short period of time and thus is convenient. As a centrifugal filter that removes a substance of a particular molecular weight from a sample by centrifugation, ultrafiltration filter kits and the like are commercialized by various life science manufacturers, and such commercialized products may be used. An example thereof is Amicon® Ultra 50K Kit (Merck).

“Adsorption” is a method of using a carrier having high affinity to an aggregation inhibitor and adsorbing an aggregation inhibitor onto the surface and/or the inside of the carrier to remove the aggregation inhibitor. An aggregating protein-containing solution or a solvent thereof is mixed with a carrier to adsorb an aggregation inhibitor to the carrier, the carrier is then separated from an aggregating protein-containing solution or a solvent thereof, and the step can be implemented. A type of a carrier may be adequately selected in accordance with a type of an aggregation inhibitor to adsorb. When an aggregation inhibitor is albumin, for example, albumin-adsorbing silica can be used as a carrier. With the use of a surface-modified carrier, alternatively, albumin can be adsorbed on the surface of a carrier based on the ion exchange principle. In addition, albumin can be adsorbed on the surface of the carrier. Specific examples of surface modification include a method of using a myristoylation reagent to modify the surface of a carrier, a method of using a palmitoylation reagent to modify the surface of a carrier, a method of using a diphenylcyclohexane compound to modify the surface of a carrier, and a method of using an antibody, an active fragment thereof, or a nucleic acid aptamer binding specifically to an aggregation inhibitor to modify the surface of a carrier.

Surface modification using a myristoylation reagent can be performed by, for example, removing a Boc group of N-Boc-N-tetradecanoyl-L-lysine [B5366] and modifying a silica, alumina, polystyrene, or quantum dot carrier with [B5366].

Surface modification using a palmitoylation reagent can be performed by, for example, modifying a silica, alumina, polystyrene, or quantum dot carrier with 1-tert-butyl 5-(N-succinimidyl)-N-palmitoyl-L-glutamate.

Surface modification using a diphenylcyclohexane compound can be performed by, for example, modifying a silica, alumina, polystyrene, or quantum dot carrier with a diphenylcyclohexane compound.

Surface modification using an antibody, an active fragment thereof, or a nucleic acid aptamer can be performed by, for example, binding an anti-actin antibody, an anti-albumin antibody, an anti-globulin antibody, or an active fragment of any thereof, an actin-binding nucleic acid aptamer, an albumin-binding nucleic acid aptamer, or a globulin-binding nucleic acid aptamer to various carriers (e.g., a resin, glass, or magnetic carrier).

Antibodies include a monoclonal antibody, a polyclonal antibody, a recombinant antibody, a synthetic antibody, and an antibody fragment. In the case of a polyclonal or monoclonal antibody, an immunoglobulin molecule of any class (e.g., IgG, IgE, IgM, IgA, IgD, or IgY) may be used. While a polyclonal or monoclonal antibody is not limited, in general, an antibody derived from a mammal, bird, or other animal may be used. Examples thereof include antibodies derived from a mouse, a rat, a guinea pig, a rabbit, a goat, a donkey, a sheep, a camel, a horse, a chicken, and a human.

A “recombinant antibody” is a chimeric antibody, a humanized antibody, or a multispecific antibody. A “chimeric antibody” comprises amino acid sequences of antibodies derived from different animals, and such antibody is prepared by substituting a variable region (a V region) of a given antibody with a V region of another antibody. An example thereof is an antibody prepared by substituting a V region of a mouse monoclonal antibody with a V region of a human antibody, and such antibody comprises a mouse-derived variable region (a V region) and a human-derived C region. A “humanized antibody” is a grafted antibody prepared by substituting complementarity determining regions (CDRs: CDR1, CDR2, and CDR3) in a variable region (a V region) of an adequate mouse antibody with CDRs of a human monoclonal antibody. In a chimeric antibody and a humanized antibody, the V regions of heavy and light chains or complementarity determining regions in the V regions of heavy and light chains are derived from a non-human animal antibody, such as a mouse antibody, and framework regions (FRs: FR1, FR2, FR3, and FR4) in the C or V region and the C regions of heavy and light chains are derived from a human antibody. Thus, an immune response to such antibody can be reduced in a human body. A “multispecific antibody” is a multivalent antibody; i.e., an antibody comprising a plurality of antigen-binding sites in a molecule, in which each antigen-binding site binds to a different epitope. An example is a bispecific antibody having 2 antigen-binding sites, such as IgG, in which each antigen-binding site binds to a different epitope.

A “synthetic antibody” is synthesized chemically or by recombinant DNA technology. Examples thereof include antibodies newly synthesized by recombinant DNA technology. Specific examples thereof include a single-chain antibody (scFv: a single chain fragment of variable region), a diabody, a triabody, and a tetrabody.

Examples of “antibody fragments” include Fab, F(ab′₂), and Fv.

An antibody may be modified. Antibody modification encompasses functional modification and labeling modification. Examples of functional modification include glycosylation, acetylation, formylation, amidation, phosphorylation, and PEGylation. Examples of labeling modification include labeling with fluorescent dyes (e.g., fluorescein. FITC, rhodamine, Texas Red, Cy3, and Cy5), fluorescent proteins (e.g., PE, APC, and GFP), enzymes (e.g., horseradish peroxidase, alkaline phosphatase, and glucose oxidase), radioactive isotopes (e.g., ³H, ¹⁴C, and ³⁵S), biotin, and (strept)avidin.

An “aptamer” is a ligand molecule that, because of its conformation, can bind strongly and specifically to a target substance and specifically inhibit functions of the target substance. A “nucleic acid aptamer” is composed of nucleic acids. Nucleic acids constituting a nucleic acid aptamer may be DNA, RNA, or DNA in combination with RNA. According to need, a nucleic acid aptamer can comprise chemically-modified nucleic acids, such as PNA, LNA/BNA, methylphosphonate DNA, phosphorothioate DNA, and 2′-O-methyl RNA.

A nucleic acid aptamer may be labeled, according to need. Any nucleic acid labeling substance known in the art can be used. Examples thereof include radioactive isotopes (e.g., ³²P, ³H, and ¹⁴C), DIG, biotin, fluorescent dyes (e.g., FITC, Texas, cy3, cy5, cy7, FAM, HEX, VIC, JOE, Rox, TET, Bodipy493, NBD, and TAMRA), and luminescent substances (e.g., acridinium ester).

In addition, albumin or globulin may be removed from a sample with the use of commercialized kits available from various life science manufacturers. Examples include AlbuVoid™ (Biotech Support Group), Albumin & IgG Depletion Spintrap™ (Cytiva), the Actin Binding Protein Spin-Down Biochem Kit™ (Cytoskeleton), and the ProMax Albumin Removal Kit (Polysciences).

“Centrifugation” is a method of molecular weight fractionation by centrifugation. This step can be implemented by subjecting an aggregating protein-containing solution or a solvent thereof to centrifugation and removing a fraction of 40 kDa or higher comprising an aggregation inhibitor. According to need, fractionation may be performed by, for example, sucrose density gradient centrifugation. As with the case of filtration, centrifugation is preferable when a molecular weight of aggregating proteins is significantly different from that of an aggregation inhibitor. As described above, centrifugation can be employed as a secondary method of filtration or adsorption and performed in combination therewith.

A step of removal may be performed a plurality of times. When it is impossible to sufficiently remove an aggregation inhibitor from an aggregating protein-containing solution or a solvent thereof in a single step, it is particularly preferable to perform a step of removal a plurality of times. In such a case, a method of removal in each step of removal may be the same with or different from a method of removal in another step.

### 2-2-2. Step of Detection

The “step of detection” comprises detecting an aggregate after the step of aggregation. In this step, an aggregate formed by aggregation of aggregating proteins and/or aggregating fragments thereof is detected visually and/or quantitatively.

As a method for detecting an aggregate, a method for detecting a peptide aggregate known in the art can be employed. In general, an aggregate can be detected. Accordingly, the presence or absence of an aggregate is detected based on the form of an aggregate by visual observation or optical microscopic observation. When aggregating proteins and/or aggregating fragments thereof are labeled, an aggregate can be detected by the method of detection based on the label. When aggregating proteins and/or aggregating fragments thereof are labeled with an optical labeling substance such as a quantum dot, for example, an aggregate may be detected by fluorescent observation using a fluorescent microscope. When a spotty fluorescent mass is observed, an aggregate can be perceived to be formed. Alternatively, the fluorescent intensity may be quantified, the quantified fluorescent intensity may be compared with the fluorescent intensity of a negative control, and presence or absence of formation of an aggregate may be detected based on the results of comparison. Alternatively, an aggregate can be detected by molecular-weight-based molecular sieving such as gel electrophoresis.

As a method for measuring the aggregated amount of aggregating proteins, a method for visualizing an aggregate of aggregating proteins, or a method for determining inhibitory activity on aggregation of aggregating proteins, for example, a method in accordance with the method of microliter-scale high throughput screening (MSHTS) of an amyloid β protein aggregation inhibitor using a quantum dot nanoprobe (Patent Document 1) may be employed. A size of aggregation of aggregating proteins that can be visualized by the aforementioned method is, for example, 0.01 to 200 μm², preferably 1 to 200 μm², and particularly preferably 10 to 150 μm².

When an aggregating protein is the amyloid β protein (e.g., the amyloid β42 protein), for example, in accordance with the MSHTS method, a quantum-dot-modified amyloid β protein (e.g., the amyloid β40 protein) is added to the solution comprising the amyloid D protein collected by the method of the first aspect, an aggregate of the amyloid D protein with the quantum-dot-modified amyloid D protein is measured using, as the indicator, the quantum dot, or an aggregate of the amyloid β protein with the quantum-dot-modified amyloid β protein is visualized based on the quantum dot.

As described in Patent Document 1, a quantum dot is a nanomaterial having a three-dimensional quantum-confinement structure, and, for example, a semiconductor quantum dot and a carbon quantum dot are known. Examples of quantum dots include semiconductor quantum dots, in particular, core-shell CdSe/ZnS quantum dots, such as Qdot® 525, Qdot545, Qdot565, Qdot585, Qdot605, Qdot655, Qdot705, and Qdot800 (Thermo Fisher Scientific).

An aggregation reaction between the amyloid β protein and the quantum-dot-modified amyloid D protein in the solution comprising the amyloid D protein collected by the method according to the first aspect is performed under conditions in which the amyloid D proteins are polymerized to form an aggregate. At the beginning of the aggregation reaction, the concentration of the amyloid β protein is approximately 1 to 100 μM, and preferably 10 to 50 μM at the final concentration thereof in the reaction solution. The concentration of the quantum-dot-modified amyloid β protein is approximately 0.005% to 0.5%, and preferably 0.01% to 0.1%, based on the concentration of the amyloid β protein.

An aggregation reaction can be performed in wells of a microplate that is generally used in fluorescent observation. In order to accurately measure the aggregated amount of the amyloid D protein, it is preferable that aggregate thickness be uniform. In this respect, it is preferable that the surface of the aggregation reaction solution be horizontal to the well bottom where the aggregate is deposited. Specifically, it is preferable that the bottom of the wells be flat. Since it is possible to measure the aggregated amount of the amyloid β protein with a reaction solution in an amount on a microliter-scale, use of, for example, a 1536-well flat bottom microplate is particularly preferable.

An aggregation reaction is performed at approximately room temperature to 37° C. and preferably at 37° C., and a reaction duration is approximately 4 to 36 hours, and preferably 12 to 24 hours. Shaking or agitation may excessively accelerate aggregate formation and it may adversely affect determination of the amount of amyloid β protein aggregation. Thus, it is preferable that an aggregation reaction be performed in a stationary state.

Subsequently, an image of fluorescence of the aggregation reaction product obtained by the aggregation reaction is obtained under exposure conditions in which the sum intensity determined based on the luminance value of pixels included in an area of interest in the fluorescent image becomes 15% to 85%.

An image of fluorescence of a quantum dot-based aggregation reaction product (aggregate) can be obtained with the use of an apparatus connected to a computer that controls operations, such as setting and regulation of image-obtaining conditions, and imaging and display of the obtained data. A typical imaging apparatus is an epifluorescence microscope equipped with a digital camera (CCD or CMOS), a container accommodating the aggregation reaction product, such as a microwell plate, is mounted thereon, an excitation light is applied thereto, fluorescence is emitted from the aggregation reaction product, and an image thereof is obtained using a CCD camera. An excitation light can be adequately determined in accordance with properties of a quantum dot used. When Qdot 605 is used as a quantum dot, for example, the wavelength of the excitation light may be shorter than 580 nm, and it may be 532 to 552 nm. As a band pass filter to selectively transmit the emitted fluorescence and obtain an image of the fluorescence, a filter that can selectively transmit a light in a wavelength band, which includes the fluorescent wavelength of the quantum dot to be used, may be selected. When Qdot 605 is used as a quantum dot, for example, a band pass filter that can selectively transmit a light in a wavelength band from 594 nm to 646 nm can be used.

A fluorescence image of an aggregation reaction product (an aggregate) is obtained by regulating the exposure to adjust the sum intensity determined in accordance with the equation 2) below on the basis of the luminance value of pixels included in a region of interest in the obtained fluorescence image to 15% to 85%.

Sum intensity (%)=(total luminance value of pixels in region of interest/total theoretical maximum luminance value of pixels in region of interest)×100 Equation 2)

Specifically, imaging under sum-intensity-based exposure control is performed in the manner described below. At the outset, a region of interest is designated in a fluorescence image of the aggregation reaction product preliminary obtained under adequate exposure conditions, and information on luminance values of pixels included therein is then obtained. A fluorescent image may be a colored or monochrome image. In the case of a colored image, an RGB value of each pixel is converted to grayscale in accordance with the equation shown below to obtain information on luminance values.

Value of luminance (Y)=0.299×R+0.587×G+0.114×B

In an 8-bit image, a value of luminance is in the range of 0 to 255. When a region of interest is an 8-bit image composed of 432×432 pixels, accordingly, the sum intensity is determined in accordance with the following equation.

Sum intensity (%)=(total luminance value of 186,624 pixels in the region of interest/(186,624×255))×100

The number of bits of a fluorescent image is preferably 8. While it is possible to use an image larger than 8 bits, image processing requires a laborious procedure. Accordingly, it is preferable that an image larger than 8 bits be converted to an 8-bit image and the sum intensity be then calculated.

With reference to the sum intensity based on the thus-calculated preliminary fluorescent image, exposure is controlled to adjust the sum intensity to a predetermined level, and imaging is then performed. Exposure can be controlled by regulating the exposure time and the camera gain (ISO sensitivity) and using a neutral-density filter and, in particular, by adequately regulating the exposure time and the camera gain (ISO sensitivity). Use of an automatic exposure control of a commercialized CCD camera is preferable. When the sum intensity calculated from the preliminary fluorescent image is lower than the predetermined level, for example, the exposure time may be prolonged and/or the camera gain may be increased, so as to increase the sum intensity. When the sum intensity calculated from the preliminary fluorescent image is higher than the predetermined level, the exposure time may be shortened and/or the camera gain may be decreased, so as to decrease the sum intensity. The sum intensity is not particularly limited, provided that it is within the range of 15% to 85%, and it is preferably adjusted within the range of 45% to 65%.

The preliminary fluorescent image can be obtained immediately before the fluorescent image of the aggregation reaction product used for evaluation is obtained. With reference to the sum intensity thereof, exposure can be feedback-controlled to adjust the sum intensity to a predetermined level. Alternatively, the preliminary fluorescent image may be obtained before evaluation, the exposure conditions to adjust the sum intensity to a predetermined level may be determined, and evaluation may then be initiated.

In a preferable embodiment, the exposure time to adjust the sum intensity to 15% to 85% is 150 ms to 1.8 s, and preferably 300 ms to 1.6 s, at sensitivity equivalent to ISO 200 (1× gain in the case of the camera with sensitivity equivalent to ISO 200). In a more preferable embodiment, the exposure time to adjust the sum intensity to 45% to 65% is 500 to 900 ms, at sensitivity equivalent to ISO 200. With the use of an automatic exposure control of a CCD camera, alternatively, the target maximum light intensity is set at 50%, and exposure is performed up to 160 ms at sensitivity equivalent to ISO 200. When the target maximum light intensity does not reach 50% within the exposure time, it is preferable that the ISO sensitivity be gradually raised to the level equivalent to 6400 (32× gain in the case of the camera with sensitivity equivalent to ISO 200) until the target maximum light intensity reaches 50%. The “target maximum light intensity” is a percentage indicating a value of luminance of the brightest pixel among the pixels in the imaging range relative to the camera's gradation. When the target maximum light intensity is designated to be 50% in a 256-level camera that can acquire 8-bit images, imaging is performed to adjust a value of luminance of the brightest pixel among the pixels in the imaging range to 128. In such a case, an acceptable level of over illumination may adequately be determined.

Subsequently, SD is calculated based on the luminance values of pixels in the region of interest in the acquired fluorescent image. As described above, the fluorescent image used herein may be a colored or monochrome image. In the case of a colored image, an RGB value of each pixel is converted to grayscale, and a colored image is preferably an 8-bit image.

In an embodiment, SD may be calculated with the use of the corrected luminance values of pixels calculated in accordance with the equation 3) shown below instead of the luminance value of pixels.

Corrected value of luminance=value of luminance×(target sum intensity/sum intensity calculated in accordance with the equation 2) Equation 3)

As a result of correction, the sum intensity values can be adjusted uniform among fluorescent images acquired under different exposure conditions. The target sum intensity can be adequately adjusted in the range of 45% to 65%.

All fluorescent images may be subjected to correction. In order to increase the value of luminance SD and more accurately measure the amount of amyloid β protein aggregation, it is preferable that a fluorescent image exhibiting a sum intensity calculated in accordance with the equation 2 that is lower than the target sum intensity be subjected to correction and SD be calculated. In the case of a fluorescent image exhibiting a sum intensity higher than the target sum intensity, it is preferable that such image be not subjected to correction and SD be calculated using an uncorrected value of luminance.

The amount of amyloid β protein aggregation is measured with the use of the value of luminance SD as the indicator based on a positive correlation between the value of luminance SD determined based on the fluorescent image of the amyloid β protein aggregate and the amount of amyloid β protein aggregation.

### 2-3. Effects

According to the method of the present invention, aggregating proteins are aggregated in a medium, culture supernatant, or the like more similar to the in vivo environment, aggregation thereof can be visualized, and the aggregated amount can be measured, which could not be implemented in the past. This enables construction of a screening technique, such as MSHTS, that can yield high accuracy in a medium or culture supernatant. Such screening technique is expected to show applicability as a tool for searching for a medicine useful for treatment or prevention of various diseases caused by aggregating proteins, including amyloidosis such as AD and Parkinson's disease.

According to the method of the present aspect, an aggregate of aggregating proteins can be visualized in the collected solution (filtrate) obtained by the method of the first aspect, and the aggregated amount of aggregating proteins can be measured. In particular, the aggregating proteins in the collected solution (filtrate) obtained by the method of the first aspect are in an environment similar to the in vivo conditions. According to the method of the present aspect, an aggregate of aggregating proteins in an environment similar to the in vivo conditions can be visualized.

According to the method of the present aspect, accordingly, preventive pharmaceutical products and functional processed products for diseases shown in Table 1 caused by various aggregating proteins can be searched in a solution in conditions more similar to the in vivo conditions (e.g., a cell secretory factor) with the use of a solution for accelerating and visualizing protein aggregation.

TABLE 1
Examples of amyloidosis

| Disease | Aggregating proteins |
|---|---|
| Alzheimer dementia | Amyloid β, tau |
| Parkinson's disease | α-Synuclein |
| Transmissible spongiform encephalopathy | Prion |
| (bovine spongiform encephalopathy) | |
| Huntington's disease | Huntingtin |
| Type II diabetes | Amylin |
| Arterial sclerosis | Apolipoprotein A1 |
| Articular rheumatism (Rheumatoid arthritis) | Serum amyloid A |
| Systemic AL amyloidosis | Immunoglobulin light chain |
| Dialysis amyloidosis | β2 Microglobulin |

### 3. Agent for Visualizing Aggregation

### 3-1. Concept

The third aspect of the present invention relates to an agent for visualizing aggregation. The agent for visualizing aggregation of the present aspect can provide a place where aggregation of aggregating proteins takes place in an environment similar to the in vivo conditions, and such agent consists of a medium or culture supernatant from which an aggregation inhibitor has been removed. With the use of the agent for visualizing aggregation of the present invention, aggregating proteins and/or aggregating fragments thereof in the solution can be aggregated, and the resulting aggregate can be visualized. In the screening system for searching for a candidate compound having inhibitory effects on aggregation of aggregating proteins, accordingly, the agent can be used as a solvent or solubilizer of the aggregating protein-containing solution.

### 3-2. Constitution

The agent for visualizing aggregation of the present aspect consists of a medium or culture supernatant from which an aggregation inhibitor has been removed. The agent for visualizing aggregation of the present aspect may be in a liquid or solid state (including powdery, granular, and particulate states). When the agent is in a solid state, the agent is dissolved in an adequate solution (e.g., water or a buffer) at the time of use.

The ingredients in the agent for visualizing aggregation of the present aspect comprise a wide variety of substances that can be contained in the medium or culture supernatant, however, an aggregation inhibitor has been removed therefrom in advance and thus is not contained in the agent.

The agent for visualizing aggregation of the present aspect may or may not comprise aggregating proteins and/or aggregating fragments thereof. In general, the agent does not comprise aggregating proteins and/or aggregating fragments thereof. According to need, any aggregating proteins and/or aggregating fragments thereof may be mixed with the agent for visualizing aggregation of the present invention to form an aggregate, and aggregation thereof can then be visualized.

### 3-3. Method of Preparation

The agent for visualizing aggregation of the present aspect can be prepared by a method for preparing a medium or culture supernatant while excluding the aggregation inhibitor, a method for removing an aggregation inhibitor from the prepared medium or culture supernatant, or these methods in combination.

An example of a method for preparing a medium or culture supernatant while excluding the aggregation inhibitor is a method for preparing a component free from an aggregation inhibitor, such as an albumin-free and/or globulin-free reagent.

An example of a method for removing an aggregation inhibitor from the prepared medium or culture supernatant is a method performed in accordance with the method described in “(1) Step of removal” in the step of aggregation of the second aspect.

The agent for visualizing aggregation prepared in the form of a liquid may be stored in that state before use, or it may be converted into a solid state and stored in that state. When the agent is stored in a liquid state, the agent is preferably stored below the freezing point, preferably at −20° C. or lower, −80° C. or lower, or in liquid nitrogen, so as to prevent various components, such as a protein, contained in the agent for visualizing aggregation from being inactivated or degraded.

A method for converting the agent for visualizing aggregation prepared in a liquid state into a solid state is not limited, and an example thereof is lyophilization. Lyophilization is a known technique and it may be performed in accordance with a conventional technique. The agent for visualizing aggregation prepared in a solid state is preferably stored at 10° C. or lower, 4° C. or lower, or 0° C. or lower.

### 3-4. Effects

The agent for visualizing aggregation of the present invention can be provided as a solvent or solubilizer of the aggregating protein-containing solution, which does not inhibit aggregation of aggregating proteins and is capable of visualizing aggregation, in, for example, a screening system for searching for a candidate compound having inhibitory effects on aggregation of any disease-causing aggregating proteins by performing MSHTS in an environment more similar to the in vivo environment.

### 4. Kit Used for the Method of the First or Second Aspect

### 4-1. Outline

The fourth aspect of the present invention relates to a kit used for the method of the first or second aspect.

### 4-2. Constitution

The fourth aspect of the present invention relates to a kit for collecting aggregating proteins (often abbreviated as a “collection kit” herein), a kit for measuring the aggregated amount of aggregating proteins (often abbreviated as a “kit for measuring aggregated amount” herein), or a kit for visualizing an aggregate of aggregating proteins (often abbreviated as a “kit for aggregation visualization” herein). The kit for measuring aggregated amount or the kit for aggregation visualization of the present aspect comprises a constitutional elements for allowing aggregating proteins and/or aggregating fragments thereof to aggregate in an environment more similar to the in vivo environment, such as a medium or culture supernatant and visualizing the aggregation. With the use of the collection kit of the present invention, aggregating proteins can be efficiently collected from an aggregating protein-containing solution while removing contaminants from the solution. With the use of the kit for measuring aggregated amount or the kit for aggregation visualization of the present invention, an aggregate of aggregating proteins and/or aggregating fragments thereof can be easily detected in a medium or culture supernatant from which an aggregation inhibitor has been removed.

The kit of the present aspect comprises, as essential components, a filtration filter and a solution for accelerating and visualizing protein aggregation and, as optional components, a wash solution, a means for aggregating protein quantification (e.g., an anti-aggregating protein antibody used in immunoassays), a means for labeling aggregating proteins (in the kit used for the method of the second aspect), labeled aggregating proteins (e.g., quantum-dot-modified amyloid D proteins), and a protocol. A protocol describes a method for using the kit of the present aspect.

### (1) Means for Removing Aggregation Inhibitor

A “means for removing an aggregation inhibitor” is a means for removing an aggregation inhibitor of 40 kDa or higher, at least one polypeptide selected from the group consisting of actin, albumin, and globulin, or a fragment thereof from a solvent of an aggregating protein-containing solution, such as a medium or culture supernatant. This means may be constituted to realize the method described in “(1) Step of removal” in the step of aggregation of the method for aggregation visualization of the second aspect. Examples thereof include a spin column equipped with an ultrafiltration filter capable of molecular weight fractionation at 40 kDa and/or a carrier adsorbing an aggregation inhibitor.

### (2) Means for Labeling Aggregating Proteins

A “means for labeling aggregating proteins” is a means for labeling aggregating proteins and/or aggregating fragments thereof to detect and visualize aggregation of target aggregating proteins and/or aggregating fragments thereof. Examples of the means include labeling substances and reagents and devices capable of modifying such labeling substances described in “(11) Labels” of the definitions of terms in the method for collecting an aggregating protein-containing solution of the first aspect. When a labeling substance is an optical label, such as a quantum dot, for example, a quantum dot and a reagent used to add a quantum dot to a peptide to modify the peptide can be used. Concerning a specific constitution, a means for labeling aggregating proteins may be equipped with adequate modifying reagents or devices in accordance with a type of a labeling substance to be used.

### (3) Labeled Aggregating Proteins

The term “labeled aggregating proteins” used herein refers to aggregating proteins and/or aggregating fragments thereof that have already been labeled. When the target aggregating proteins and/or aggregating fragments thereof are designated when preparing an aggregating protein-containing solution with the use of a medium or culture supernatant from which an aggregation inhibitor has been removed, the proteins and the like are labeled and included in that state in the kit of the present aspect. The label is as described in “(11) Labels” of the definitions of terms in the method for collecting an aggregating protein-containing solution of the first aspect.

### (4) Other Constituents

In addition to the constituents described above, the kit for aggregation visualization of the present aspect can optionally comprise other constituents. Examples of other constituents include, but are not particularly limited to, a variety of substances that are necessary or useful to visualize aggregation of aggregating proteins. Specific constituents may adequately be determined in accordance with, for example, types of aggregating proteins. Examples include a medium or culture supernatant from which an aggregation inhibitor has or has not been removed, a labeled secondary antibody, an enzyme substrate necessary to detect a label, a wash buffer, a sample diluent, and instructions.

### 5. Method of Production Characterized by Forming an Aggregate of Aggregating Proteins

### 5-1. Concept

The fifth aspect of the present invention relates to a method of production characterized by forming an aggregate of aggregating proteins (the method is often abbreviated to the “method of aggregate production” herein). The method of aggregate production of the present aspect comprises incubating the entirely or partially labeled target aggregating proteins and/or aggregating fragments thereof in an aggregating protein-containing solution from which an aggregation inhibitor has been removed and forming an aggregate thereof. According to the method of aggregate production of the present aspect, an aggregate consisting of aggregating proteins and/or aggregating fragments thereof can be produced and obtained. The resulting aggregate can be used in the screening system for searching for a candidate compound having effects of dissociating aggregation that can dissociate the aggregate.

### 5-2. Method

The method of aggregate production of the present aspect comprises, as essential steps, a step of aggregation and a step of confirmation and, as an optional step, a step of collection. Hereafter, the steps are described.

### 5-2-1. Step of Aggregation

The “step of aggregation” of the present aspect is basically in accordance with the step of aggregation of the method for aggregation visualization of the second aspect. As with the step of aggregation of the second aspect, aggregating proteins and/or aggregating fragments thereof constituting the target aggregate are incubated under predetermined conditions in an aggregating protein-containing solution from which an aggregation inhibitor has been removed to form an aggregate. In this case, the aggregating proteins and/or aggregating fragments thereof used to form an aggregate are entirely or partially labeled. A method of labeling is also in accordance with “(11) Labels” of the definitions of terms in the method for collecting an aggregating protein-containing solution of the first aspect. A method of labeling is preferably modification with a labeling substance, although the method is not particularly limited thereto. A labeling substance is not limited, and labeling with an optical label, and, in particular, a quantum dot, is preferable.

Types of aggregating proteins are not limited, and proteins associated with diseases caused by protein aggregation, such as Aβ or tau protein associated with AD, are preferable.

### 5-2-2. Step of Confirmation

A “step of confirmation” comprises detecting the aggregating proteins aggregated in a solution after the step of aggregation and confirming formation of an aggregate. In the step of confirmation, formation of a product, an aggregate, is to be confirmed, and an aggregate is detected to confirm the formation. Accordingly, the step of confirmation is basically in accordance with the step of detection of the method for aggregation visualization of the second aspect.

### 5-2-3. Step of Collection

When an aggregate is confirmed in the step of confirmation, the aggregate is collected in the “step of collection.” The step of collection is an optional step in the method of aggregate production of the present aspect.

An aggregate can be collected by a method known in the art by which a protein aggregate can be collected. Examples include filtration, adsorption, centrifugation, and two or more thereof in combination. Since specific methods are in accordance with the method described in “(1) Step of removal” in the step of aggregation of the method for aggregation visualization of the second aspect, description thereof is omitted herein.

The collected aggregate can be resuspended in a solvent according to need. Examples of solvents that can be used include physiological saline, a buffer, a medium, and a culture supernatant. A solvent is preferably a medium or culture supernatant similar to the in vivo environment. A medium or culture supernatant used herein is preferably a medium or culture supernatant from which an aggregation inhibitor has been removed.

### EXAMPLES

Hereafter, the present invention is described in greater detail with reference to Examples, although the technical scope of the present invention is not limited to these Examples.

### 1. Material and Method

### Example 1

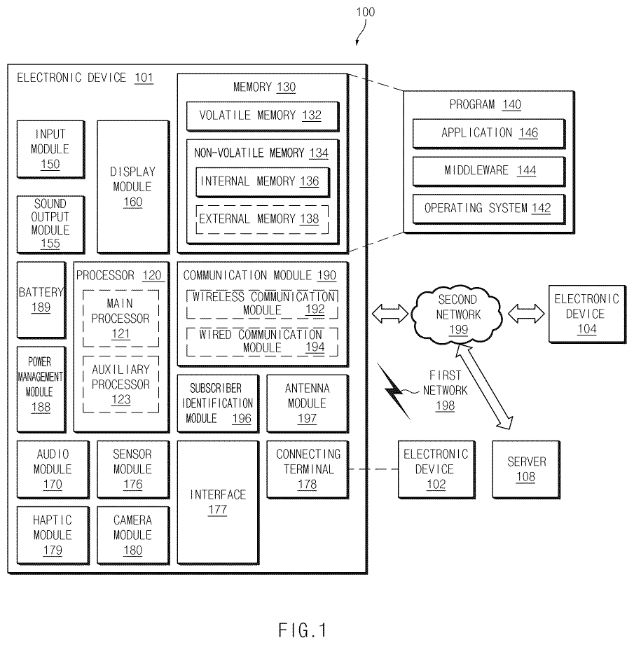

A mixture comprising an Nb medium (NbM) prepared by diluting B27 (Life Technologies Corporation) to 50-fold with Neurobasal medium (without phenol red) (Life Technologies Corporation) and the amyloid β42 protein (Aβ₄₂) adjusted to the final concentration of 50 μM in DMSO was prepared (hereafter, referred to as a “simulated solution”). A filtration filter device (Amicon Ultra-0.5 Ultracel-50 Membrane) was mounted on a 1.5-ml tube, and 400 μl of MilliQ water was added dropwise to the filtration filter with the use of MilliQ water and the simulated solution at room temperature (15° C. to 25° C.). The 1.5-ml tube was centrifuged at room temperature (15° C. to 25° C.) and 14,000×g for 10 minutes, the filtrate was discarded, and 400 μl of MilliQ water was added dropwise to the filter again. The tube was further centrifuged at room temperature (15° C. to 25° C.) and 14,000×g for 10 minutes, the filtrate was discarded, the empty tube was centrifuged at room temperature (15° C. to 25° C.) and 14,000×g for 2 minutes, the filtration filter device was mounted on another 1.5-ml tube, 500 μl of the simulated solution was added dropwise to the filtration filter, the tube was centrifuged at room temperature (15° C. to 25° C.) and 14,000×g for 30 minutes, and the filtrate was collected. The concentration of Aβ₄₂ contained in the simulated solution and that in the filtrate were measured using the human β amyloid ELISA Kit (FUJIFILM Wako Pure Chemical Corporation). The concentration of Aβ₄₂ in the simulated solution was designated as 100%, the concentration of Aβ₄₂ in the filtrate was divided by the concentration of Aβ₄₂ in the Aβ₄₂-containing simulated solution, the obtained value was converted to a percentage, and the Aβ₄₂ residual ratio was calculated to be 53.8% (FIG. 1). For the purpose of accuracy evaluation, the standard curve was prepared using NbM containing artificial Aβ₄₂ at 50 μM, 25 μM, 10 μM, 5 μM, 2.5 μM, and 1 μM, the recovery rate was compared with that of the standard solution in the human Aβ ELISA Kit, and the rate was found to be 92.3%.

### Example 2

Blocking 1 to Blocking 6 were prepared below. Blocking 1 indicates a 5% BSA-containing PBS solution, Blocking 2 indicates a solution comprising Tween 20 adjusted to the final concentration of 0.1% in a 5% BSA-containing PBS solution, Blocking 3 indicates a 5% BSA-containing PBS solution, Blocking 4 indicates a solution comprising Tween 20 adjusted to the final concentration of 0.1% in a 5% BSA-containing PBS solution, Blocking 5 indicates a 0.4% BSA-containing PBS solution, and Blocking 6 indicates a solution comprising Tween 20 adjusted to the final concentration of 0.1% in a 0.4% BSA-containing PBS solution.

As in the case of Example 1, the filtration filter device (Amicon Ultra-0.5 Ultracel-50 Membrane) was mounted on a 1.5-ml tube, and 400 μl of MilliQ water was added dropwise to the filtration filter, and the tube was centrifuged at room temperature (15° C. to 25° C.) and 14,000×g for 10 minutes.

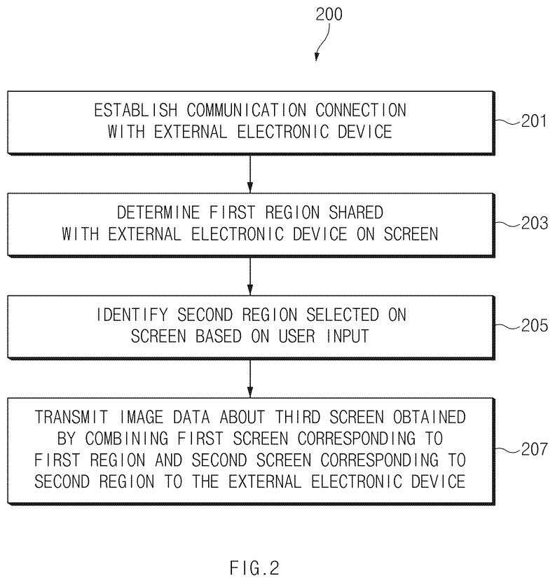

The filtrate was discarded, and 500 μl each of Blocking 1 to Blocking 6 were added dropwise to the filtration filter, followed by incubation. Incubation conditions are as follows: Blocking 1. Blocking 2, and Blocking 3 were each allowed to stand at room temperature (15° C. to 25° C.) for 2 hours, and Blocking 4, Blocking 5, and Blocking 6 were each allowed to stand under refrigeration (4° C.±2° C.) for 18 hours. After blocking, the 1.5-ml tube was centrifuged at room temperature (15° C. to 25° C.) and 14,000×g for 2 minutes, the filtrate was discarded, and the tube was centrifuged at room temperature (15° C. to 25° C.) and 14.000×g for 10 minutes. The filtrate was discarded, 400 μl of MilliQ water was added dropwise to the filter, the tube was centrifuged at room temperature (15° C. to 25° C.) and 14,000×g for 10 minutes, the filtrate was discarded again, 400 μl of MilliQ water was added dropwise to the filter, the tube was centrifuged at room temperature and 14,000×g for 10 minutes, the filtrate was discarded, and the empty tube was centrifuged at room temperature (15° C. to 25° C.) and 14,000×g for 2 minutes. The filtration filter device was mounted on another 1.5-ml tube, 500 μl of the simulated solution was added dropwise to the filtration filter, the tube was centrifuged at room temperature (15° C. to 25° C.) and 14,000×g for 30 minutes, and the filtrate was collected. As described above, the concentration of Aβ₄₂ contained in the simulated solution and that in the filtrate were measured using the human β amyloid ELISA Kit (FUJIFILM Wako Pure Chemical Corporation). The residual ratios of Aβ₄₂ were calculated based on the concentrations of Aβ₄₂ in the filtrates collected under the conditions of Blocking 1 to Blocking 6. As a result, the composition and the conditions of Blocking 2 were found to exhibit the highest residual ratio of Aβ₄₂ (93.8%) (FIG. 2).

### Example 3

In Example 2 above, the highest residual ratio of Aβ₄₂ was achieved under the conditions of Blocking 2. Accordingly, whether or not a high Aβ collection ratio would be maintained with the use of a culture supernatant containing the amyloid β protein (AD) that have been actually secreted by cells (including Aβ, such as Aβ₄₂ and the amyloid β38 protein (Aβ₃₈)) was examined. At the outset, a culture supernatant containing secreted Aβ (Aβ secreted by cells) was prepared in the manner described below.

A coating solution prepared by diluting Matrigel (Corning) to 25- to 50-fold with DMEM (Life Technologies Corporation) or a solution prepared by diluting the SureBond-XF solution (Axol) to 200-fold with PBS (Mg—, Ca—) were added at 1 ml/well to a 6-well culture plate, and incubation was performed for 1 to 3 hours. The iPS-cell-derived neural precursor cells exhibiting the survival rate of 70% or higher thawed in an incubator were suspended in a culture solution comprising NbM supplemented with 5 μM Y-27632 (Nacalai Tesque) or a culture solution comprising NMMs medium of Neural Maintenance Medium (Axol) and Neural Maintenance Medium Supplement (Axol)) supplemented with 5 μM Y-27632, cells were seeded at the density of 8 to 16-10⁵ cells/well (0.84 to 1.7×10⁵/cm²) on the culture plate from which the coating solution had been removed, and culture was performed at 37° C. in the presence of 5% CO₂ for 2 days. Thereafter, the culture supernatant was removed by suction, a fresh culture solution returned to room temperature was added at 2 ml/well, and culture was performed at 37° C. in the presence of 5% CO₂ for an additional 2 days. The iPS-cell-derived neural precursor cells 5 days after thawing were treated with Accutage (STEMCELL Technologies) for 3 to 5 minutes, culture was dissociated into single cells by pipetting, 5 ml of DMEM/F-12 was added, the cells were transferred to a centrifuge tube, and the supernatant was removed by centrifugation. To the precipitated cells, a culture solution comprising NbM supplemented with 5 μM Y-27632 or a culture solution comprising NMMs medium supplemented with 10 μM Y-27632 was added, and cells were dispersed by pipetting to prepare a cell suspension. The cells were seeded at the density of 8.0 to 16×10¹ cells/well (0.84 to 1.7×10⁵/cm²) on the culture plate from which the coating solution had been removed, and culture was performed at 37° C. in the presence of 5% CO₂ for 2 days. Thereafter, the culture supernatant was removed by suction, fresh NbM returned to room temperature was added at 2 ml/well, and culture was performed at 37° C. in the presence of 5% CO₂ for 2 days. On the ninth day, the iPS-cell-derived neural precursor cells were treated with Accutage (STEMCELL Technologies) for 3 to 5 minutes, culture was dissociated into single cells by pipetting, 5 ml of DMEM/F-12 was added, the cells were transferred to a centrifuge tube, and the supernatant was removed by centrifugation. To the precipitated cells, a culture solution comprising NbM supplemented with 5 μM Y-27632 or a culture solution comprising NMMs medium supplemented with 10 μM Y-27632 was added, cells were dispersed by pipetting to prepare a cell suspension, the cells were seeded at the density of 8 to 16×10⁵ cells/well (0.84 to 1.7×10⁵/cm²) on the culture plate from which the coating solution had been removed, and culture was performed at 37° C. in the presence of 5% CO₂. On the following day, the supernatant was removed from the culture plate, the content of the culture plate was exchanged with NbM+Shh medium supplemented with the Sonic Hedgehog (Shh) protein (final concentration: 100 ng/ml) at 2 ml/well, and culture was performed at 37° C. in the presence of 5% CO₂ for 3.5 days. Further, a half amount of the culture supernatant was collected from the culture plate, the NbM+Shh medium was added at 2 ml/well, and culture was performed at 37° C. in the presence of 5% CO₂ for 3.5 days. The culture supernatant collected from the culture plate was collected in a centrifuge tube, the tube was centrifuged at 300×g for 3 minutes, the culture supernatant was transferred to another centrifuge tube while retaining 1 ml of the culture supernatant from the bottom, and the centrifuge tube was then cryopreserved at −20° C. (the culture supernatant on Day 7). Thereafter, a half amount of the medium was collected every 3.5 days, fresh NbM was added at 2 ml/well, and culture was performed at 37° C. in the presence of 5% CO₂.

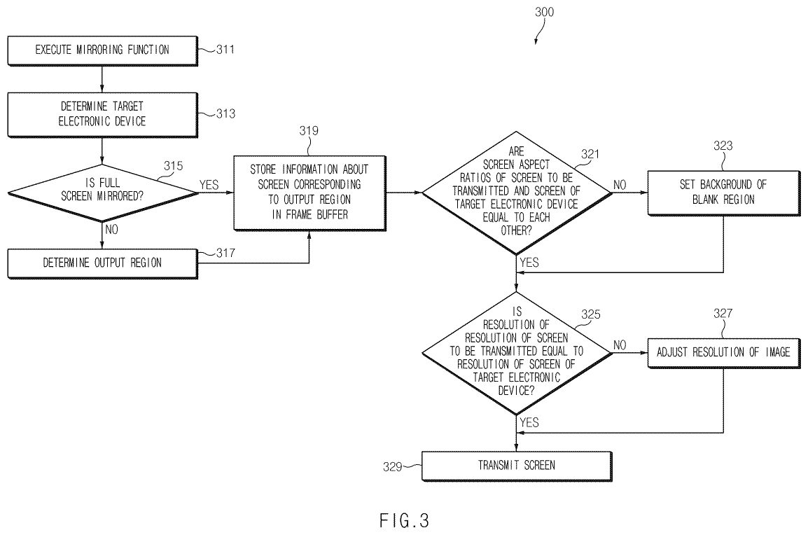

The culture supernatant of the cells at least 14 days after the addition of Shh was collected. The concentration of Aβ contained in the collected culture supernatant was measured using the human β amyloid ELISA Kit (FUJIFILM Wako Pure Chemical Corporation) and found to be 43.6 μM (FIG. 3). With the use of this culture supernatant, whether or not removal of Aβ would be reduced with the use of a culture supernatant containing Aβ that had been actually secreted by cells was examined under the conditions of Blocking 2 where the highest residual ratio of Aβ₄₂ was achieved in Example 2. For comparison, the residual ratio of Aβ without blocking was calculated in the same manner as in Example 1. The results demonstrate that a high residual ratio of Aβ (79.3%) could be maintained with the culture supernatant containing AD secreted by cells under the conditions of Blocking 2 (FIG. 3).

### Example 4

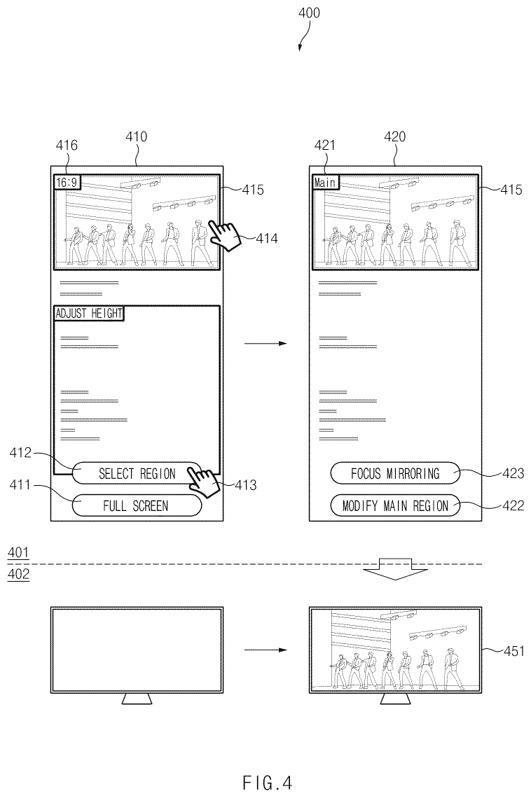

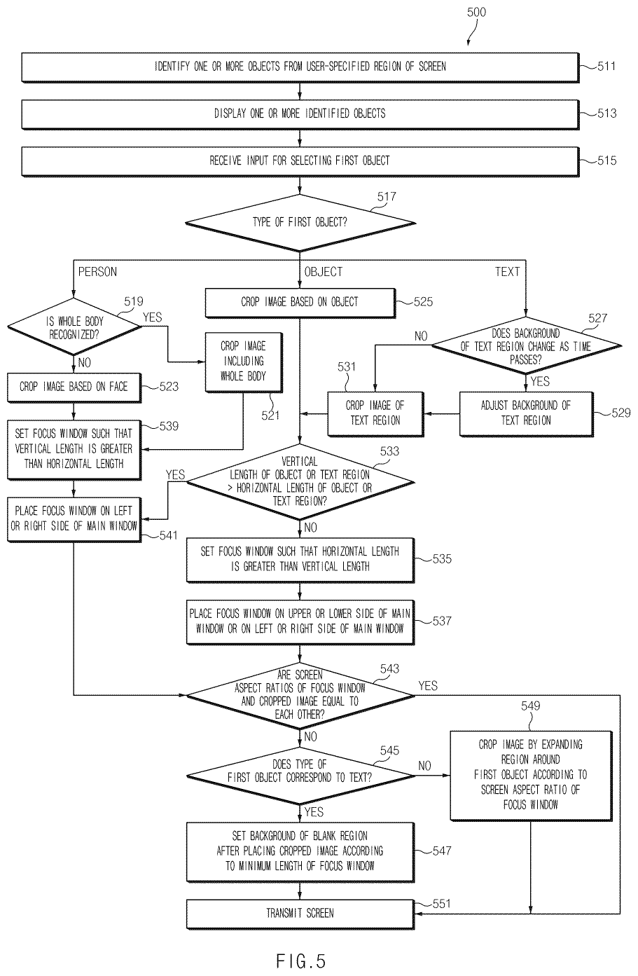

It was demonstrated in Example 3 above that a high residual ratio of Aβ would be achieved when the filtrate collected from the culture supernatant containing Aβ actually secreted by cells under the conditions of Blocking 2. Accordingly, whether or not it is possible to visualize aggregation of the amyloid β protein in such filtrate was examined. The quantum-dot-modified amyloid 40 protein (QD-Aβ₄₀) was dissolved in the filtrate collected in Example 3, the solution was added to and mixed with the 1 mM Aβ₄₂ solution, and the filtrate collected in Example 3 comprising 50 μM Aβ₄₂ and 50 nM QD-Aβ₄₀ was prepared. The 150 mM rosmarinic acid (RA) solution was diluted with the filtrate collected in Example 3 to five levels of concentration (3,000 μM, 600 μM, 120 μM, 24 μM, and 4.8 μM). Aβ₄₂ and QD-A$40 were diluted with the filtrate collected in Example 3 to prepare a mixture of 50 μM Aβ₄₂ and 50 nM QD-Aβ₄₀. The mixture of Aβ₄₂ and QD-Aβ₄₀ was mixed with the rosmarinic acid (RA) solution at 1:1 to prepare reaction solutions comprising RA at the final concentrations of 1,500 μM, 300 μM, 60 μM, 12 μM, and 2.4 μM. A solution comprising DMSO instead of RA was prepared as a negative control. These reaction solutions were incubated at 37° C. for 24 hours, and aggregated Aβ was observed under a fluorescent microscope. As a result, none of the solutions were sufficient in terms of sensitivity of SD values to perform quantitative analysis of aggregated Aβ (FIG. 4).

### Example 5

On the basis of Example 4 above, the composition of the blocking solutions and the treatment conditions were examined again, and Blocking (Buffer) 8 to Blocking (Buffer) 12 were additionally prepared as follows. Blocking 8 indicates a solution comprising Tween 20 adjusted to the final concentration of 0.1% in a 0.4% BSA-containing PBS solution, Blocking 9 indicates a 0.4% BSA-containing PBS solution, Blocking 10 indicates a solution comprising Tween 20 adjusted to the final concentration of 0.1% in a 0.4% B27-containing PBS solution, Blocking 11 indicates a 0.4% B27-containing PBS solution, and Blocking 12 indicates a solution comprising Tween 20 adjusted to the final concentration of 0.1% in PBS. As in the case of Example 2, the filtration filter device (Amicon Ultra-0.5 Ultracel-50 Membrane) was mounted on a 1.5-ml tube, and 400 μl of MilliQ water was added dropwise to the filtration filter device, and the tube was centrifuged at room temperature (15° C. to 25° C.) and 14,000×g for 10 minutes.

The filtrate was discarded, 400 μl of MilliQ water was added dropwise to the filter again, and the tube was centrifuged at room temperature (15° C. to 25° C.) and 14,000×g for 10 minutes. The filtrate was discarded, and 500 μl each of the Blocking solutions (PBS, 8 to 12) were added dropwise to the filtration filter, followed by incubation. Incubation conditions are as follows: Blocking PBS, 8 to 12 were each allowed to stand at room temperature (15° C. to 25° C.) for 2 hours. After blocking, the tube was centrifuged, and the filtrate was discarded, followed by centrifugation at room temperature (15° C. to 25° C.) and 14,000×g for 10 minutes. The filtrate was discarded, 400 μl of MilliQ water was added dropwise to the filter, the tube was centrifuged at room temperature (15° C. to 25° C.) and 14,000×g for 10 minutes, the filtrate was discarded again, 400 μl of MilliQ water was added dropwise to the filter, the tube was centrifuged at room temperature and 14,000×g for 10 minutes, the filtrate was discarded, and the empty tube was centrifuged at room temperature (15° C. to 25° C.) and 14,000×g for 2 minutes. The filtration filter device was mounted on another 1.5-ml tube, 500 μl of the simulated solution containing 50 μM Aβ₄z was added dropwise to the filtration filter, the tube was centrifuged at room temperature (15° C. to 25° C.) and 14,000×g for 30 minutes, and the filtrate was collected. As described above, the concentration of Aβ₄₂ contained in the simulated solution and that in the filtrate were measured using the human β amyloid ELISA Kit (FUJIFILM Wako Pure Chemical Corporation). The concentration of Aβ₄₂ in the simulated solution was designated as 100%, and the concentration of Aβ₄₂ in each filtrate was calculated. As a result, the composition and the conditions of Blocking 12 were found to exhibit the highest residual ratio of Aβ₄₂ (83.9%. FIG. 5). In addition, 50 μM Aβ₄₂ solutions were prepared using the filtrates collected by blocking procedures (PBS, 8 to 12), and a 50 nM QD-Aβ₄₀ solution was prepared using the filtrate that had collected 11.1 μM QD-Aβ₄₀. The Aβ₄₂ solutions were mixed with the QD-Aβ₄₀ solution at 1:1, followed by incubation at 37° C. for 24 hours. As a result of observation of aggregated AD under a fluorescent microscope, it was found possible to visualize aggregated Aβ with PBS, Blocking 8, Blocking 10, Blocking 11, and Blocking 12 (FIG. 5).

In particular, Blocking 12; i.e., a 0.1% Tween 20 solution, was found to exhibit the highest sensitivity (SD value) as the solution for accelerating and visualizing protein aggregation.

With the use of Blocking 12, accordingly, a 50 μM Aβ₄₂ solution was prepared from the filtrate collected in the same manner as in Example 2, and QD-Aβ₄₀ was diluted in the collected filtrate to adjust the concentration of QD-Aβ₄₀ to 50 nM. Thus, Solution B was prepared. With the use of Solution B, reaction solutions comprising RA at the final concentrations of 1,500 μM, 300 μM, 60 μM, 12 μM, and 2.4 μM were prepared in the same manner as in Example 4. A reaction solution comprising DMSO instead of RA was prepared as a negative control. These reaction solutions were incubated at 37° C. for 24 hours, aggregated Aβ was observed under a fluorescent microscope, and aggregated AD was quantified. The results demonstrate that, at the RA concentration of 46.0±7.4 μM or less, Aβ accounting for approximately 50% of the whole would not be aggregated. It was thus found that use of the solution for accelerating and visualizing protein aggregation would enable calculation of inhibitory activity on Aβ aggregation in the filtrate. It was found necessary that the sensitivity of the SD value be 60% or higher relative to that of PBS designated as 100% for quantification of aggregated AD.

### 2. Results and Discussion

When contaminants were removed from an aggregating protein-containing medium (aggregating proteins may be artificially added or secreted by cells/tissue), disadvantageously, aggregating proteins were also removed from the medium (FIG. 1).

Accordingly, treatment with non-adsorbing solutions that reduce non-specific adsorption of various proteins (e.g., BSA/PBS and BSA/PBS plus 0.1% Tween 20) was examined, types and other conditions of non-adsorbing solutions/wash solutions were optimized, and removal of aggregating proteins from the collected solutions was thus reduced (FIG. 2). However, when contaminants were removed from a culture supernatant containing Aβ secreted by nerve cells by the optimized method, the amount of secreted Aβ to disappear simultaneously was found to be reduced (FIG. 3).

When QD-Aβ₄₀ and artificial Aβ₄₂ were allowed to be present in the collected solution, it was impossible to visualize aggregated AD by fluorescence observation (FIG. 4).

The present inventors conducted concentrated studies and found a solution for accelerating and visualizing protein aggregation (Buffer 12: a 0.1% Tween 20 solution), which can efficiently remove contaminants from an aggregating protein-containing solution and visualize aggregated Aβ by fluorescence observation in the presence of QD-Aβ₄₀ and artificial Aβ₄₂ in the collected solution (FIG. 5).

### Example 6

### Confirmation of Aggregate Formation Depending on Solvent Type

### (Objective)

By microliter-scale high throughput screening (MSHTS), formation of an aggregate of the amyloid β proteins with the use of various solvents is examined.

### (Method)

### (1) Preparation of Amyloid β Protein Solution

As aggregating proteins, the amyloid β proteins (AD) were used. In the manner described below, a 1 mM Aβ solution was prepared. To 5 mg of Aβ (human, 1-42) (Peptide Institute, Inc.), 5 ml of HFIP (1,1,1,3,3,3-hexafluoro-2-propanol) (FUJIFILM Wako Pure Chemical Corporation) was added to prepare a suspension, and the suspension was then allowed to stand at room temperature for 1 hour. Thereafter, the suspension was ultrasonicated at 25° C. and 43 kHz for 10 minutes for monomerization, and the resultant was allowed to stand in a clean bench for 24 hours to vaporize HFIP. Thereafter, the resultant was dissolved in 1,071 μl of DMSO to obtain a 1 mM Aβ solution. The Aβ solution was dispensed into 1.5-ml tubes in an amount of 256 μl each and stored at −80° C. before use.

The Aβ solution prepared herein is hereafter referred to as “Aβ42.”

### (2) Preparation of Quantum Dot-Modified Amyloid β Protein (QDAβ)

Aβ was modified with a quantum dot (QD) in the manner described below to prepare a quantum dot-modified amyloid β protein (QDAβ). The method of modification was as described below.

Qdot™605 ITK™ amino (PEG) Quantum Dots (8 μM, 125 μl) were introduced into two 1.5-ml tubes, and the tubes were centrifuged at 10,000×g and 4° C. for 1 minute. The supernatants thereof were transferred to centrifugal tubes (VIVASPIN6), and 4,500 μl PBS was added thereto. Centrifugation was continued at 4° C. and 3,800×g until the total amount in the two tubes was reduced to about 50 μl or less, and the filtrates were discarded. After PBS was filled, centrifugation was performed again until the amount was reduced to 50 μl. The QD solutions obtained were introduced into a tube to adjust the total amount to approximately 180 μl, 10 mM sulfo-EMCS (20 μl) was added, and the resultant was allowed to stand at room temperature for 1 hour to prepare QD-EMCS.

Two columns for desalting were prepared, approximately 800 μl of resin was introduced into each thereof, and centrifugation was performed at 1,000×g and 4° C. for 1 minute. PBSE (300 μl) was loaded onto each column and centrifugation was performed at 1,000×g and 4° C. for 1 minute. This procedure was performed two times to prepare desalting columns.

After the preparation of QD-EMCS, 20 μl of 100 mM K-glutamic acid was added in order to inactivate an unreacted N-hydroxysuccinimide group contained in the solution, and the resultant was allowed to stand at room temperature for 10 minutes.

The QD-EMCS (110 μl) was allowed to impregnate into the center of each of two desalting columns, and 15 μl of PBSE was added as a stacker. After the columns were subjected to centrifugation at 1,000×g and 4° C. for 2 minutes, filtrates (desalted QD-EMCS) of the two desalting columns were gathered together. To the desalted QD-EMCS obtained, 20 μl of 1.0 mM Cys-Aβ/DMSO were added and mixed therein, and the resulting mixture was then allowed to stand at room temperature for 1 hour.

After the preparation of QD-EMCS, 20 μl of 100 mM 2-mercaptoethanol was added in order to inactivate an unreacted maleimide group contained in the solution, and the resultant was allowed to stand at room temperature for 10 minutes.

The sample solution was transferred in an amount of 145 μl each to two VIVASPIN6 columns, 4,500 μl of water was added thereto, and the resultants were centrifuged at 3,800×g and 4° C. for 17 minutes. The filtrates were discarded, and the obtained solutions were introduced into a tube to adjust the total amount of the obtained solutions to approximately 140 μl.

Water (300 μl) was introduced into a desalting column, centrifugation was performed at 1,000×g and 4° C. for 1 minute, and this procedure was performed two times. The sample solution (70 μl) was allowed to impregnate the desalting column, and 15 μl of stacker water was added. Centrifugation was performed at 1,000×g and 4° C. for 2 minutes to obtain QDAβ of interest. The completed QDAβ and unmodified QD (Qdot™605 ITK™amino(PEG) Quantum Dots) were each diluted to 8-fold with QD/QDAβ:ultrapure water (0.4 μl:2.8 μl), and the concentration was measured using the NanoDrop.

### (3) Preparation of Evaluation Solution

The following evaluation solutions to confirm aggregate formation were prepared for each solvent.

### BSA/PBS Solution

A solution prepared by dissolving BSA (Wako) at 50 mg/ml in PBS

### NBM Solution

A solution prepared by adding B-27® (Gibco) at 0.5% v/v to the Neurobasal medium

### DFBM Solution

A solution prepared by adding B-27® (Gibco) at 0.5% v/v to the DMEM/F12 medium (Thermo Fisher Scientific)

### (4) Aggregate Formation

An Aβ solution containing 50 nM QDAβ and 50 μM Aβ42 at levels comparable to twice the final concentration was prepared. To 3 μl of each evaluation solution, 3 μl of the Aβ solution was added to prepare sample solutions. The sample solutions were centrifuged at 10,000×g for 2 minutes and fractionated at 5 μl/well on the plate. Subsequently, centrifugation was performed at 1,530×g for 5 minutes using a plate centrifuge. The time before aggregation was designated as “0 hour,” the occurrence of aggregation was examined, and incubation was then performed at 37° C. for 24 hours. Thereafter, images of aggregates in the sample solutions were obtained using inverted microscopes (Nikon TE2000/Olympus DP72).

### (Results)

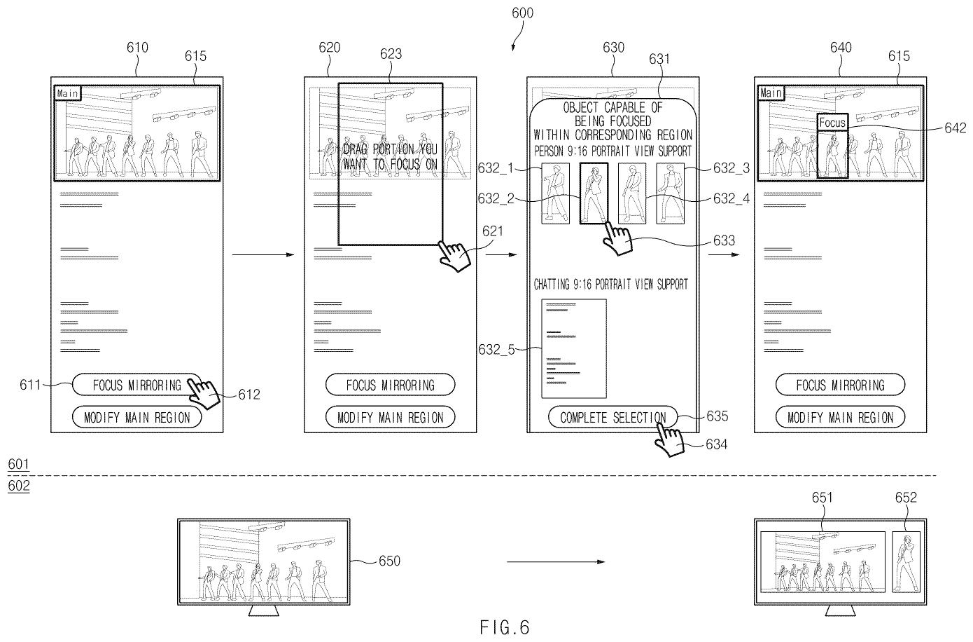

FIG. 6 shows the results. In the sample solution comprising PBS used in the conventional method as a solvent. Aβ aggregation was observed 24 hours after the initiation of incubation as shown in FIG. 6 b. In the sample medium comprising, as a solvent, a sample solution comprising PBS supplemented with BSA (c), the NBM solution (d), or the DFBM solution (e), in contrast, aggregation was not observed 24 hours later. Thus, it was deduced that an aggregation inhibitor inhibiting Aβ aggregation is present in the medium.

### Example 7

### Confirmation of Aβ Aggregate Formation by Removal of Aggregation Inhibitor (1)

### (Objective)

In Example 6, the presence of a substance that would inhibit Aβ aggregation in the medium was deduced. Thus, whether or not aggregated Aβ could be visualized by removing an Aβ aggregation inhibitor from the sample solution was examined.

### (Method)

Four types of ultrafiltration filters with different fractionation sizes (Amicon Ultra; 3 kDa: UFC500324, 10 kDa: UFC501024, 50 kDa:UFC505096, and 100 kDa:UFC510024, Merck) were mounted on a 1.5-ml tube included in the kit. The NBM solution (500 μl) was added dropwise to each ultrafiltration filter, and centrifugation was performed at room temperature (20° C. to 25° C.) and 14,000×g for 10 minutes. The filtrate was collected and mixed with a mixture of 50 nM QD-Aβ solution (quantum-dot-modified amyloid β) and 50 μM As at 1:1, the resultant was incubated at 37° C. for 24 hours, and aggregated AD was observed under an inverted microscope.

### (Results)

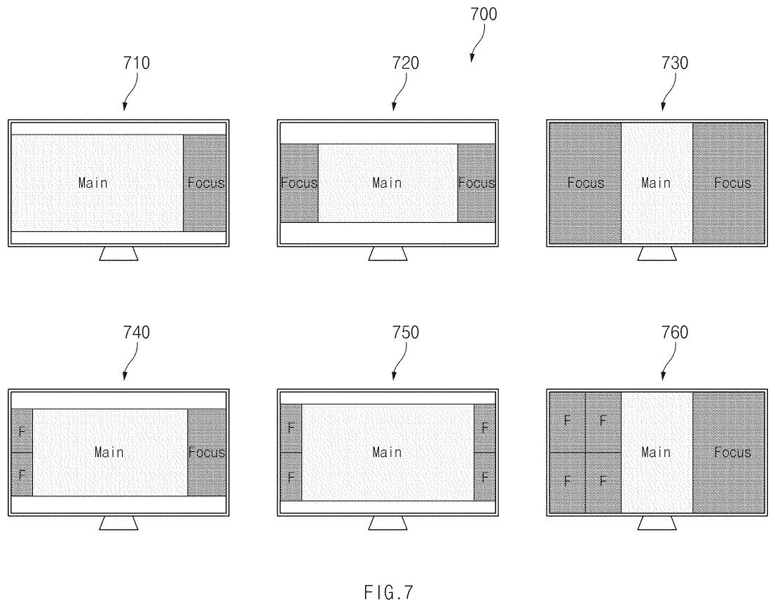

FIG. 7 shows the results. The results of observation demonstrate that AR aggregation non-uniformly occurred in the filtrate collected with a 100 kDa ultrafiltration filter, Aβ aggregation was not sufficiently quantified, and EC50 could not be calculated. In the filtrates collected with 3 kDa, 10 kDa, and 50 kDa ultrafiltration filters, in contrast, uniform Aβ aggregation was observed. The results demonstrate that the Aβ aggregation inhibitor comprises a substance of a molecular weight that cannot pass through a 50 kDa ultrafiltration filter; i.e., a substance that is removed by a 50 kDa ultrafiltration filter.

### Example 8

### Evaluation of Aggregation Inhibitor

### (Objective)

In Example 7, an Aβ aggregation inhibitor was found to be removed by a 50 kDa ultrafiltration filter. As described above, a substance of a molecular weight of 50 kDa or lower can be actually removed even if the nominal molecular weight limit (NMWL) is 50 kDa. In order to confirm components removed from the NBM solution and the DFBM solution, accordingly, molecular weight fractionation by SDS-PAGE and CBB staining were performed.

### (Method)

(1) Preparation of a Solution from which the Aβ Aggregation Inhibitor has been Removed

An ultrafiltration filter (Amicon Ultra; 50 kDa: UFC505096; Merck) was mounted on a 1.5-ml tube included in the kit. The NBM solution and the DFBM solution (500 μl each) were added dropwise to the ultrafiltration filter, and centrifugation was performed at room temperature (20° C. to 25° C.) and 14,000×g for 10 minutes. The filtrates were collected, and the NBM solution (removed) and the DFBM solution (removed) from which the Aβ aggregation inhibitor has been removed were prepared.

### (2) Preparation of SDS-PAGE Gel

SDS-PAGE gel was prepared using a 30% acrylamide stock solution (29% acrylamide, 1% N,N′-methylenebisacrylamide), a 1.5 M Tris-HCl (pH 8.8) solution, a 10% SDS (sodium dodecyl sulfate) solution, a 10% APS (ammonium persulfate) solution, and TEMED (N,N,N′,N′-tetramethylethylenediamine). A 10% acrylamide running gel solution (final concentration: 9.7% acrylamide, 0.3% N,N′-methylenebisacrylamide, 0.38 M Tris-HCl (pH 8.8), 0.1% SDS, 0.033% APS, and 0.05% TEMED) was injected into a space between two glass plates, water was then injected thereinto, the resultant was allowed to stand for 1 hour to prepare 10% acrylamide running gel. Water on the top was removed, a stacking gel solution prepared with the use of a 30% acrylamide stock solution, a 0.5 M Tris-HCl (pH 6.8) solution, a 10% SDS solution, a 10% APS solution, and TEMED (final concentration: 4.6% acrylamide, 0.15% N,N′-methylenebisacrylamide, 0.125 M Tris-HCl (pH 6.8), 0.1% SDS, 0.033% APS, and 0.05% TEMED) was injected thereinto, a comb was inserted therein, and the resultant was allowed to stand for 1 hour to prepare 10% acrylamide gel.

### (3) Preparation of Electrophoresis Sample

The NBM solution, the NBM solution (removed), the DFBM solvent, the DFBM solution (removed), and 50 mg/ml BSA/PBS (25 μl each) were each mixed with 25 μl of a 2× sample buffer (100 mM Tris-HCl (pH 6.8), 4% SDS, 12% 2-mercaptoethanol, 20% glycerol, 0.01% bromophenol blue), and the resultants were heated on a heat block (95° C., FG-01N, FastGene) for 3 minutes to prepare electrophoresis sample solutions.

### (4) SDS-PAGE

The electrophoresis sample solutions of the NBM solution, the NBM solution (removed), the DFBM solvent, and the DFBM solution (removed) (10 μl each) and the electrophoresis sample solution of 50 mg/ml BSA/PBS (1 μl) were injected into lanes of the 10% acrylamide gel prepared in (2), and electrophoresis was performed using a power unit (BP-9, BIO CRAFT) at 20 mA for 75 minutes. As a molecular weight marker, 10 μl of CLEARLY Stained Protein Ladder (Code No. 3454A, Takara Bio) was used.

After electrophoresis, the gel was collected, soaked in a CBB solution (Nacalai Tesque) for 20 minutes, washed with 7.5% acetic acid for 30 minutes, and heated with the microwave approximately 3 times. An image of the destained gel was obtained.

### (Results)

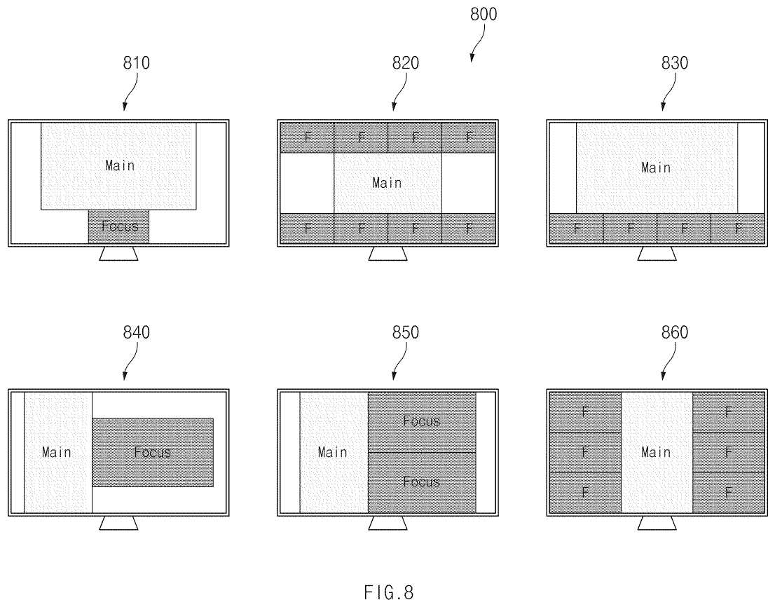

FIG. 8 shows the results. The results of observation demonstrate that proteins at around 42 kDa, 50 kDa, and 150 kDa indicated by arrows observed in the NBM solution (lane 2) and the DFBM solution (lane 4) have been removed from the NBM solution (removed) (lane 3) and the DFBM solution (removed) (lane 5). Based on the presence of a major band at around 50 kDa in bovine serum albumin (BSA) (lane 6) and the results shown in FIG. 6 c, the component of the Aβ aggregation inhibitor was deduced to be albumin with a molecular weight of approximately 50 kDa.

The NBM solution or the DFBM solution contains a large quantity of globulin or actin, the molecular weight of globulin is approximately 150 kDa, and the molecular weight of actin is 42 kDa. In SDS-PAGE shown in FIG. 8, a band observed at around approximately 150 kDa is considered to be globulin, and a band observed at around approximately 42 kDa is considered to be actin. Thus, globulin and actin are deduced to be components of an Aβ aggregation inhibitor. The above results demonstrate that an aggregation inhibitor can be removed by removing a substance having a molecular weight of 40 kDa to 250 kDa, 40 kDa to 240 kDa, 40 kDa to 230 kDa, 40 kDa to 220 kDa, or 40 kDa to 210 kDa.

### Example 9

### Confirmation of Aβ Aggregate Formation by Removal of Aggregation Inhibitor (2)

### (Method)

In Example 7, images of aggregated AD formed in the filtrates collected after filtration through various filters were analyzed using image analysis software Image J and configurations of aggregates were automatically detected (including the use of thresholding). Thereafter, 10 aggregates determined to be composed of single particles were randomly selected for each condition, and areas, perimeters, and Feret's diameters of the aggregates were calculated. In analysis using Image J. obtained images were imported into Image J, and configurations of aggregates were detected in the Adjust, Color Threshold mode in the Image tab. With the use of the Wand tool, 10 of single particles were randomly selected and applied to the Tools, ROI manager in the Analyzer tab, for analysis of the configurations of aggregates. The Measure tab in the ROI manager was pressed, the “Area,” “Perimeter,” and “Feret's Diameter” of each particle were calculated, the calculated values were averaged to prepare charts. The Feret's diameter is the longest distance between any two points connected to each other on the outer perimeter of the selected area.

### (Results)

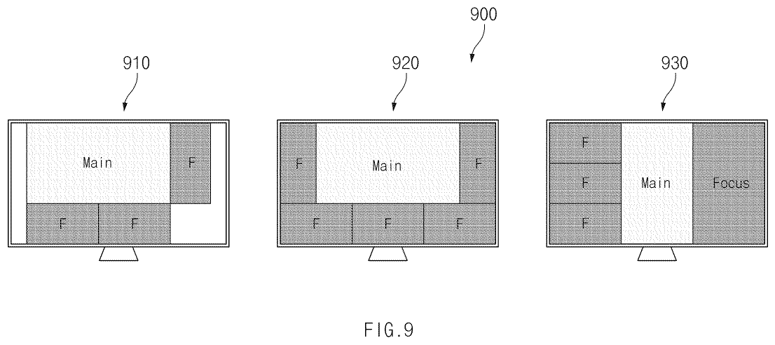

FIG. 9 shows the results. The results demonstrate that the Aβ aggregates in only the filtrate that had been filtrated through a 100 kDa ultrafiltration filter and collected were larger and less uniform compared with the filtrates filtrated through other ultrafiltration filters in terms of all of A (Area), B (Perimeter), and C (Feret's Diameter). It was thus found impossible to use the filtrate filtered through a 100 kDa ultrafiltration filter and collected for calculation of EC50.

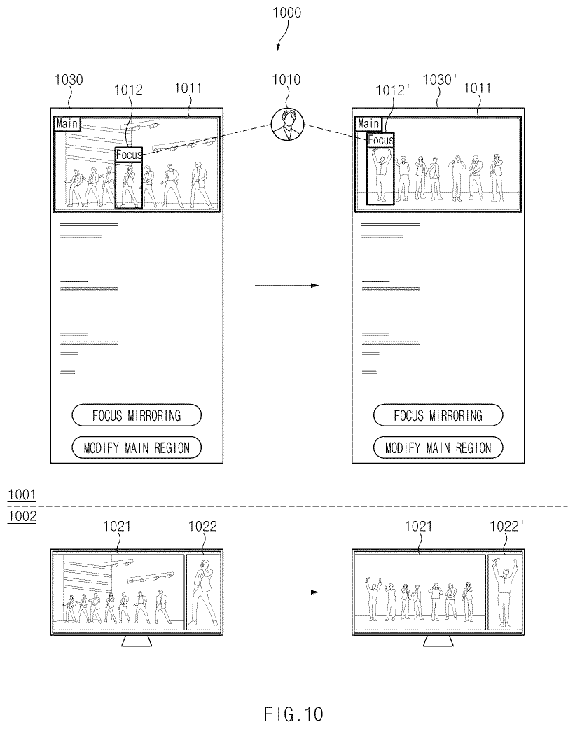

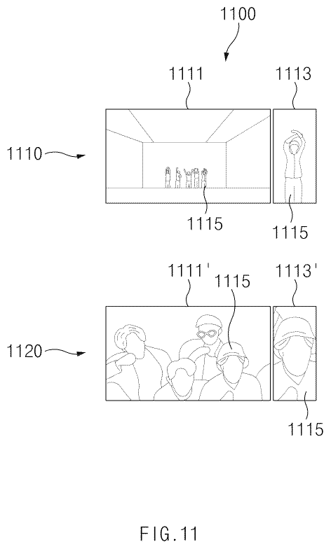

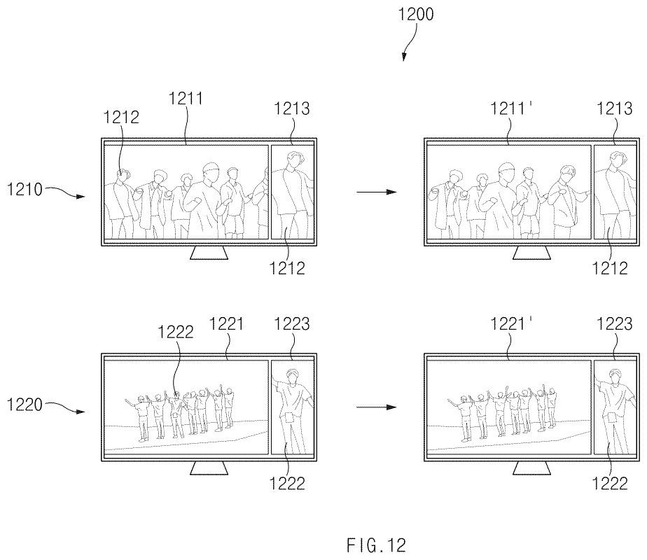

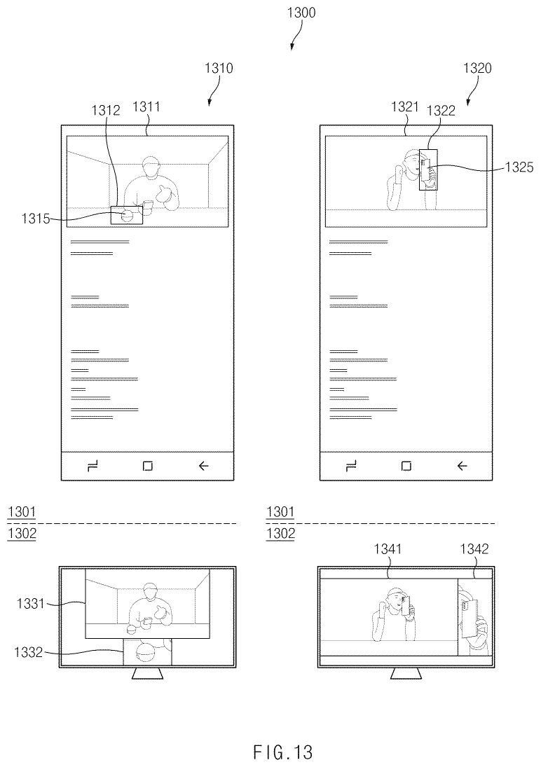

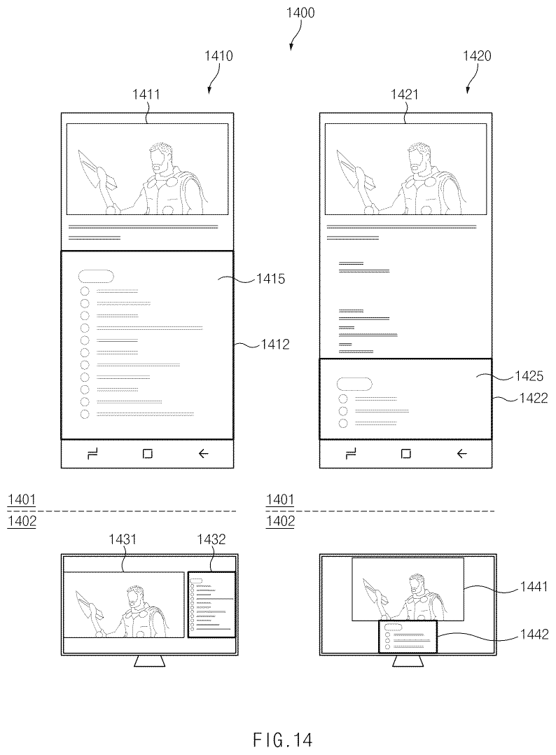

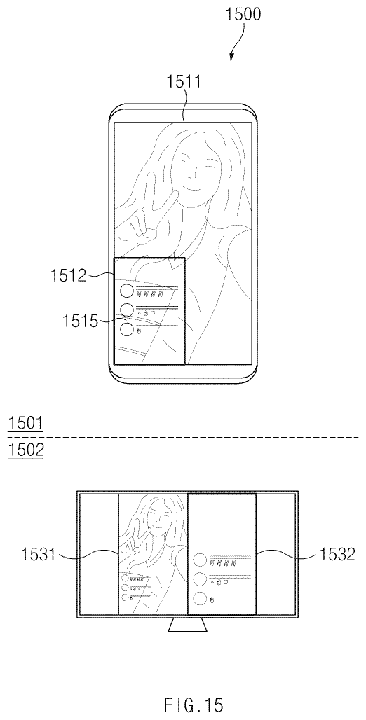

FIG. 10 shows a concept of a screening technique utilizing the above results. A culture supernatant of culture of disease model cells is collected, and aggregating proteins in the culture supernatant are aggregated and visualized by the method of the present invention. With the addition of candidate substances exerting inhibitory activity on aggregation such as various compounds and extracts at a given concentration at the time of visualization, EC₅₀ can be calculated. This enables selection of candidate substances that actually exert inhibitory activity on aggregation. Since this screening technique can be automated, high-throughput screening can be performed.

All publications, patents, and patent applications cited herein are incorporated herein by reference in their entirety.

1. A method for collecting an aggregating protein-containing solution comprising:
a first step of allowing a solution for accelerating and visualizing protein aggregation to pass through a filtration filter; and
a second step of allowing an aggregating protein-containing solution to pass through the filtration filter through which the solution for accelerating and visualizing protein aggregation has passed.

2. The method according to claim 1, which further comprises, before the first step, a step of pretreatment for allowing a wash solution to pass through aid filtration filter.

3. The method according to claim 1, which further comprises, before the second step, a step of quantifying aggregating proteins in said aggregating protein-containing solution.

4. The method according to claim 1, which further comprises, after the second step, a step of quantifying aggregating proteins in a filtrate.

5. The method according to claim 1, wherein the filtration filter is an ultrafiltration filter with a nominal molecular weight limit (NMWL) of 50 kDa or lower.

6. The method according to claim 1, wherein the solution for accelerating and visualizing protein aggregation comprises a surface-active substance of 50 kDa or lower.

7. The method according to claim 1, wherein the solution for accelerating and visualizing protein aggregation is an aqueous solution containing Tween 20.

8. The method according to claim 7, wherein the concentration of Tween 20 is 0.1% to 10% in the aqueous solution.

9. The method according to claim 1, wherein the aggregating protein-containing solution comprises a buffer, medium, or culture supernatant.

10. The method according to claim 1, wherein the aggregating protein-containing solution comprises a cell metabolite, cell secretory factor, inorganic substance, or organic acid.

11. The method according to claim 1, wherein the aggregating protein-containing solution comprises a disease-associated protein.

12. The method according to claim 11, wherein the disease is Alzheimer's disease and the protein associated therewith is the amyloid β protein or tau protein.

13. The method according to claim 1, wherein a rate of aggregating protein collection determined by the equation 1) below is over 60%,
Rate of aggregating protein collection (%)=quantitative value of aggregating proteins after second step/quantitative value of aggregating proteins before second step×100 Equation 1).

14. A method for measuring an aggregated amount of aggregating proteins comprising measuring an aggregated amount of the aggregating proteins in the aggregating protein-containing solution collected by the method according to claim 1.

15. A method for visualizing an aggregate of aggregating proteins comprising visualizing an aggregate of aggregating proteins in the aggregating protein-containing solution collected by the method according to claim 1.

16. The method according to claim 15, wherein visualization of an aggregate of aggregating proteins comprises:
a step of aggregation comprising incubating the entirely or partially labeled aggregating proteins and/or aggregating fragments thereof in the collected aggregating protein-containing solution and allowing the aggregating proteins and/or aggregating fragments thereof to aggregate; and
a step of detection comprising detecting an aggregate of the aggregating proteins and/or aggregating fragments thereof,
wherein, from the collected aggregating protein-containing solution, an aggregation inhibitor of 40 kDa or higher that inhibits aggregation of the aggregating proteins and/or aggregating fragments thereof is removed.

17. The method according to claim 16, wherein the aggregation inhibitor of 40 kDa or higher has a molecular weight of 250 kDa or lower.

18. The method according to claim 16, wherein the aggregation inhibitor of 40 kDa or higher is at least one polypeptide selected from the group consisting of actin, albumin, and globulin or a fragment thereof.

19. The method according to claim 16 wherein the label is an optical label.

20. The method according to claim 19, wherein the optical label is a quantum dot.

21. A kit for collecting aggregating proteins from an aggregating protein-containing solution by the method according to claim 1, which comprises a filtration filter and a solution for accelerating and visualizing protein aggregation.

22. (canceled)

23. (canceled)

24. (canceled)
